(19) United States
(12) Patent Application Publication
(10) Pub. No.: US 2011/0178072 A1
(43) Pub. Date: Jul. 21, 2011

(54) STEREOSPECIFICITY OF METHYLSULFINYL REDUCTION

(76) Inventors: Vadim Gladyshev, Lincoln, NE (US); Byung Cheon Lee, Lincoln, NE (US)

(21) Appl. No.: 13/055,400

(22) PCT Filed: Jul. 23, 2009
(86) PCT No.: PCT/US09/51572

§ 371 Date: Mar. 30, 2011

Related U.S. Application Data
(62) Division of application No. 61/135,704, filed on Jul. 23, 2008.

(51) Int. Cl. A61K 31/541; A61K 31/437; A61K 31/415; A61K 31/16

(52) U.S. Cl. 514/225.5; 514/303; 514/395; 514/608

(57) ABSTRACT

This disclosure relates to compositions and methods of use involving compounds (e.g., drugs) containing methylsulfinyl moieties. For example, a compound may be administered in an excess of either the R- or S-epimer of the methylsulfinyl moiety based on whether the compound exhibits higher biological activity when the methylsulfinyl moiety is present in the methylsulfinyl-oxidized form or the methylsulfide-reduced form.

### CROSS-REFERENCE TO RELATED APPLICATIONS

This application claims priority to U.S. Provisional Application Ser. No. 61/135,704, filed on Jul. 23, 2008, which is incorporated by reference in its entirety herein.

### FEDERALLY SPONSORED RESEARCH OR DEVELOPMENT

The U.S. Government has certain rights in this invention pursuant to Grant No. AG-021518-06 awarded by the National Institutes of Health.

### TECHNICAL FIELD

This disclosure relates to compositions and methods of use for compounds (e.g., drugs) containing methylsulfinyl moieties.

### BACKGROUND

A number of drugs (such as those used in cancer treatment), metabolites (for example, methionine sulfoxides) and natural compounds (for example, from broccoli and other plants) contain methylsulfinyl moieties. The oxidized sulfur atom in these compounds is asymmetric, i.e., there are two epimers for each compound (R- and S-epimers). Typically, the drugs are administered as a mixture of the two epimers.

Methionine sulfoxide reductases (Msrs) can reduce methionine sulfoxides to methionine (Met). For example, the methylsulfinyl moiety in methionine sulfoxide is converted to methylsulfide by several classes of Msrs. Other compounds, for example drugs or natural compounds, containing methylsulfinyl functional groups could function as substrates for Msrs following administration. The pathway for the reduction of compounds having methylsulfinyl moieties in mammals, however, has not been well studied.

### SUMMARY

This disclosure is based on the discovery that free compounds having a methylsulfinyl moiety can be enzymatically reduced to the methylsulfide by certain species (e.g., mammals) only when the methylsulfinyl moiety is present as the S-epimer (FIG. 1).

Accordingly, provided herein is a method of treating a subject with a compound comprising a methylsulfinyl moiety, the method including determining whether the compound comprising the methylsulfinyl moiety exhibits higher biological activity when the methylsulfinyl moiety is present in the methylsulfinyl-oxidized form or the methylsulfide-reduced form. The subject can then be contacted with a composition having the compound in an excess amount of the R-epimer relative to the S-epimer if the methylsulfinyl-oxidized form exhibits higher biological activity, or a composition comprising the compound in an excess amount of the S-epimer relative to the R-epimer if the methylsulfide-reduced form exhibits higher biological activity.

Further provided herein is a method for treating a subject with a drug having a methylsulfinyl moiety. In some embodiments, the method includes determining whether the drug having the methylsulfinyl moiety exhibits higher biological activity when the methylsulfinyl moiety is present in the methylsulfinyl-oxidized form or the methylsulfide-reduced form. The drug can then be administered, to the subject in a composition comprising the drug in an excess amount of the R-epimer relative to the S-epimer if the methylsulfinyl-oxidized form exhibits higher biological activity, or a composition comprising the drug in an excess amount of the S-epimer relative to the R-epimer if the methylsulfide-reduced form exhibits higher biological activity.

In some embodiments, the drug can be, for example, enoximone; pergolide; lincomycin; thiethylperazine; fensulfothion; nifuratel; albendazole; modafinil; captodiame; sulfinpyrazone; clindamycin; thiocolchicoside; omeprazole; flosequinan; dimethylsulfoxide; sulmazole; triclabendazole; mesoridazine; oxisuran; and sulindac. The drug can be administered with a pharmaceutically acceptable carrier or diluent. In some embodiments, the pharmaceutically acceptable carrier or diluent has a methylsulfinyl moiety if the methylsulfinyl-oxidized form exhibits higher biological activity. In some embodiments, the pharmaceutically acceptable carrier or diluent does not have a methylsulfinyl moiety if the methylsulfide-reduced form exhibits higher biological activity.

In some embodiments, the amount of the drug in the R-epimer form is at least 75% by weight compared to the S-epimer. In some embodiments, the amount of the drug in the R-epimer form is at least 90% by weight compared to the S-epimer. In some embodiments, the amount of the drug in the S-epimer form is at least 75% by weight compared to the R-epimer. In some embodiments, the amount of the drug in the S-epimer form is at least 90% by weight compared to the R-epimer.

This disclosure further provides a method of treating a subject with a drug comprising a methylsulfinyl moiety, the method including determining whether the drug comprising the methylsulfinyl moiety exhibits higher biological activity when the methylsulfinyl moiety is present in the methylsulfinyl-oxidized form or the methylsulfide-reduced form. The subject can then be administered a composition having the drug and a pharmaceutically acceptable carrier or diluent having a methylsulfinyl moiety, if the methylsulfinyl-oxidized form exhibits higher biological activity, or a composition comprising the drug and a pharmaceutically acceptable carrier or diluent lacking a methylsulfinyl moiety, if the methylsulfide-reduced form exhibits higher biological activity.

Also provided herein is a method for increasing the shelf-stability of a drug having a methylsulfinyl moiety. The method can include determining whether the drug comprising the methylsulfinyl moiety exhibits higher biological activity when the methylsulfinyl moiety is present in the methylsulfinyl-oxidized form or the methylsulfide-reduced form. Once a determination has been made, a composition can be formulated comprising the drug in an excess amount of the R-epimer relative to the S-epimer if the methylsulfinyl-oxidized form exhibits higher biological activity and an oxidant, or the composition can be formulated having the drug in an excess amount of the S-epimer relative to the R-epimer if the methylsulfide-reduced form exhibits higher biological activity and a reductant.

An oxidant can be, for example, hydrogen peroxide, hypochlorous acid, urea peroxide, sodium perborate tetrahydrate, sodium percarbonate, sodium perborate, sodium peroxide, sodium periodate, calcium peroxide, and mixtures thereof. A reductant can be, for example, dithiothreitol (DTT), a thioredoxin, sodium dithionite, sodium bisulphite, ascorbic acid, sodium ascorbate, calcium ascorbate, palmityl-DL-ascorbic acid, propyl gallate, octyl gallate, dodecyl gallate, butylhydroxyanisole gallate and butylhydroxytoluene gallate, formamidine sulphinic acid, stannous ion, Fe(II), Cu(I), erythrobate, α-tocopherol, γ-tocopherol, δ-tocopherol, oxalic acid, formic acid, and mixtures thereof.

This disclosure further provides a method for increasing the in vivo activity of a drug having a methylsulfinyl moiety in a subject. The method can include determining whether the drug comprising the methylsulfinyl moiety exhibits higher biological activity when the methylsulfinyl moiety is present in the methylsulfinyl-oxidized form or the methylsulfide-reduced form. In some embodiments, the subject can then be administered an oxidant and a composition comprising the drug in an excess amount of the R-epimer relative to the S-epimer if the oxidized form exhibits higher biological activity. In some embodiments, the subject can then be administered a reductant and a composition comprising an excess amount of the S-epimer relative to the R-epimer if the reduced form exhibits higher biological activity.

In some embodiments, the oxidant or the reductant is administered before or after the drug. In some embodiments, the oxidant or the reductant and the drug are administered together. In some embodiments, the oxidant or reductant is formulated into the composition comprising the drug.

Compositions are also provided. In some embodiments, a composition is provided, wherein a drug comprising a methylsulfinyl moiety exhibits higher biological activity when the methylsulfinyl moiety is present in the methylsulfinyl-oxidized form, having the drug in an excess amount of the R-epimer relative to the S-epimer; and an oxidant. In some embodiments, a composition is provided, wherein a drug comprising a methylsulfinyl moiety exhibits higher biological activity when the methylsulfinyl moiety is present in the methylsulfide-reduced form, having the drug in an excess amount of the S-epimer relative to the R-epimer; and a reductant.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing the metabolism of methionine (Met) and methionine sulfoxide (Met sulfoxide) in mammalian cells.

FIG. 2A illustrates the growth of SK-Hep1 hepatocytes on Met and Met sulfoxide media; FIG. 2B shows the consumption of Met in Met medium (diamonds), methionine-S-sulfoxide (Met-SO) in a racemic methionine sulfoxide (Met-RSO) medium (squares), methionine-R-suilfoxide (Met-RO) in Met-RSO medium (triangles), Met-SO in Met-SO medium (circles), and Met-RO in Met-RO medium (stars) at 0, 24, 48, 72, and 96 hours.

FIG. 3 illustrates an HPLC analysis of consumption of Met and Met sulfoxides from growth media with upper panels showing growth at zero hours and lower panels showing the growth of cells after 96 hours for each combination. FIG. 3A shows Met-SO and Met-RO signals in Met-RSO medium; FIG. 3B shows Met signal in Met medium; FIG. 3C shows Met-SO signal in Met-SO medium; and FIG. 3D shows Met-RO signal in Met-RO medium.

FIG. 4 details a characterization of MsrA-knockdown SK-Hep1 cells: FIG. 4A shows a Western blot analysis of MsrA in MsrA-knockdown SK-Hep1 cells (lane 1) and control SK-Hep1 cells (lane 2); FIG. 4B illustrates MsrA-knockdown SK-Hep1 cells grown in media containing Met (closed diamonds), Met-RO (closed circles), Met-SO (closed triangles), or Met-RSO (closed squares).

FIG. 5 details multiple sequence alignment of yeast fRMsr and its orthologs in bacteria and eukaryotes.

FIG. 6 shows a characterization of SK-Hep1 cells expressing yeast fRMsr. FIG. 6A details a Western blot analysis of SK-Hep1 cells stably expressing yeast His-tagged fRMsr (lane 1) and control SK-Hep1 cells (lane 2) with anti-His antibodies; FIG. 6B shows yeast fRMsr-transfected SK-Hep1 cells grown in media containing Met (closed diamonds), Met-RO (closed circles), Met-SO (closed triangles), or Met-RSO (closed squares); and FIG. 6C shows the resistance of fRMsr-expressing SK-Hep1 cells to hydrogen peroxide treatment.

FIG. 7 Morphology of SK-Hep1 cells grown on sulfoxide media: FIG. 7A shows an image of SK-Hep1 cells grown in the Met-SO medium; FIG. 7B shows an image of SK-Hep1 cells grown in Met-RO medium; and FIG. 7C shows an image of SK-Hep1 cells stably expressing yeast fRMsr grown in Met-RO medium.

FIG. 8 details Western blot analysis of MsrA (FIG. 8A), metabolic labeling with ⁷⁵Se (FIG. 8B), CBS (FIGS. 8C and 8D), and ATF3 (FIGS. 8E and 8F) expression in SK-Hep1 cells grown in Met and Met sulfoxide media.

FIG. 9 illustrates the growth of SK-Hep1 cells in selenium-deficient media. FIGS. 9A and 9B shows the growth of cells grown in Met-free medium supplemented with Met (diamonds), Met-RO (circles), Met-SO (triangles), Met-RSO (squares), or with no addition of these compounds (stars); FIG. 9C details the growth of cells under various conditions including 10% FBS and 0.1 mM Met (closed diamonds); 10% FBS, 0.1 mM Met, and 100 nM Se (open diamonds); insulin, transferrin, and 0.1 mM Met (closed squares); insulin, transferrin, 0.1 mM Met, and 100 nM Se (open squares); insulin, transferrin, and 0.1 mM Met-RSO (closed triangles); and insulin, transferrin, 0.1 mM Met-RSO, and 100 nM Se (open triangles).

FIG. 10 details an analysis of Met-SO and Met-RO in mouse plasma.

FIG. 11 illustrates an HPLC analysis of Met-SO and Met-RO in mouse plasma from wild type (FIG. 11A), heterozygous MsrA knockout (FIG. 11B), homozygous MsrA knockout (FIG. 11C), and selenium-deficient (SD) (FIG. 11D) mice. Elution of sulfoxides is indicated by arrows.

FIG. 12 shows an HPLC analysis of the DTT-S-sulforaphane adduct incubated with buffer (as control) (FIG. 12A), mouse MsrB2 (FIG. 12B), and mouse MsrA (FIG. 12C).

FIG. 13 shows an HPLC analysis of the DTT-R-sulforaphane adduct incubated with buffer (as control) (FIG. 13A), mouse MsrB2 (FIG. 13B), and mouse MsrA (FIG. 13C).

FIG. 14 illustrates a reduction of mesoridazine by mouse MsrA (FIG. 14A), mouse MsrB2 (FIG. 14B), and yeast fRMsr (FIG. 14C). FIG. 14D details the specific activities of various Msrs for the reduction of mesoridazine.

FIG. 15 illustrates the reduction of triclabendazole sulfoxides by mouse MsrA (FIG. 15A), mouse MsrB2 (FIG. 15B), and yeast fRMsr (FIG. 15C). FIG. 15D details the specific activities of various Msrs for the reduction of triclabendazole sulfoxide.

FIG. 16 illustrates the reduction of sulmazole by mouse MsrA (FIG. 16A), mouse MsrB2 (FIG. 16B), and yeast fRMsr (FIG. 16C). FIG. 16D details the specific activities of various Msrs for the reduction of sulmazole.

FIG. 17 illustrates the reduction of DMSO by mouse MsrA (FIG. 17A), mouse MsrB2 (FIG. 17B), and mouse MsrB1-Cys (FIG. 17C). FIG. 17D details the specific activities of various Msrs for the reduction of DMSO.

### DETAILED DESCRIPTION

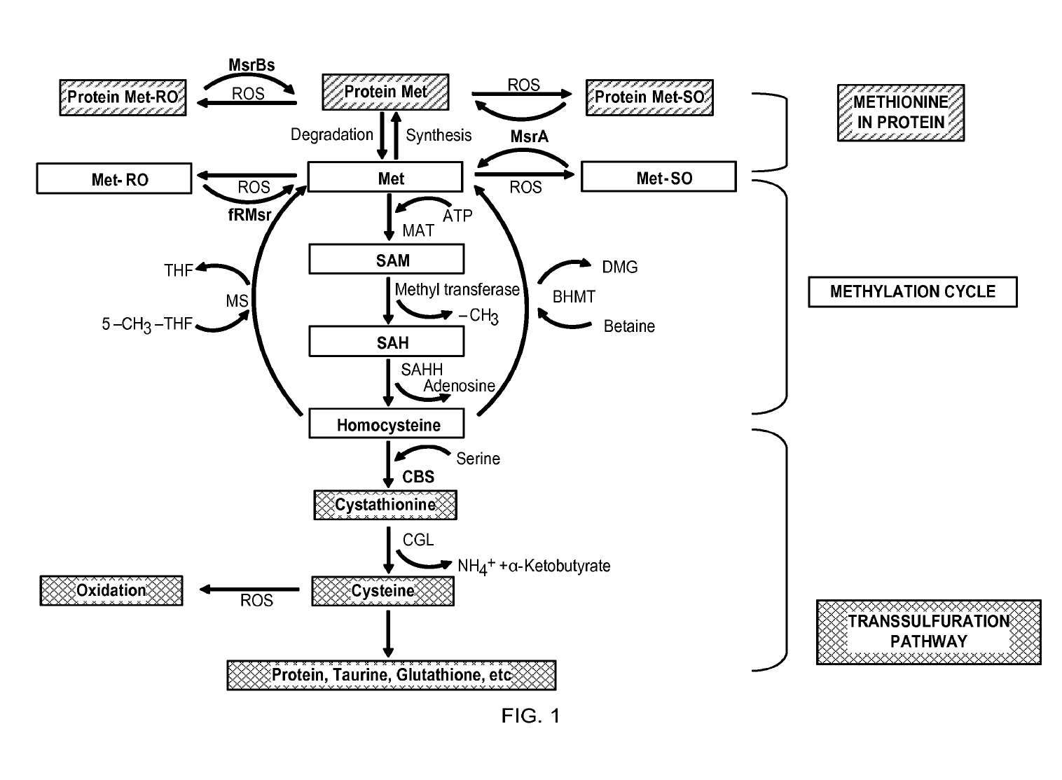

This disclosure is based on the discovery that free compounds having a methylsulfinyl moiety can be enzymatically reduced to the methylsulfide by certain species (e.g., mammals) only when the methylsulfinyl moiety is present as the S-epimer (FIG. 1) (Lee B. C., Le D. T., Gladyshev V. N. (2008) J Biol Chem., 283(42), 28361-28369). There are three known classes of methionine sulfoxide reductase enzymes. Methionine sulfoxide reductase A (MsrA) reduces both free and protein-incorporated methionine-S-sulfoxides. Methionine sulfoxide reductase B (MsrB) specifically reduces methionine-R-sulfoxide residues in proteins, but cannot reduce free methionine-R-sulfoxides. A third enzyme, free methionine-R-sulfoxide reductase (fRMsr) reduces free methionine-R-sulfoxides, but cannot reduce protein-incorporated methionine-R-sulfoxide residues. Thus, a combination of three enzymes (MsrA, MsrB, and fRMsr) can reduce both free and protein-incorporated sulfoxides, as well as both S- and R-epimers of these compounds. However, while some organisms, such as E. coli and S. cerevisiae contain all three classes of Msr enzymes, the inventors have discovered that animals, including mammals, only have MsrA and MsrB enzymes. Those animals lacking the fRMsr enzyme are unable to convert free R-methylsulfinyl epimers to methylsulfides. Such knowledge provides an avenue for increased drug efficacy that can apply to both currently used drugs and future drugs containing methylsulfinyl moieties.

Accordingly, provided herein is a method for treating a subject with a drug comprising a methylsulfinyl moiety. The method can include determining whether the drug comprising the methylsulfinyl moiety exhibits higher biological activity when the methylsulfinyl moiety is present in the methylsulfinyl-oxidized form or the methylsulfide-reduced form; and administering, to the subject, a composition comprising the drug in an excess amount of the R-epimer relative to the S-epimer if the methylsufinyl-oxidized form exhibits higher biological activity, or a composition comprising the drug in an excess amount of the S-epimer relative to the R-epimer if the methylsulfide-reduced form exhibits higher biological activity.

As used herein, a “subject” (as in the subject of the treatment) refers to animals, including both mammals and non-mammals. Mammals include, for example, humans; non-human primates, e.g. apes and monkeys; cattle; horses; sheep; rats; mice; pigs; and goats. Non-mammals include, for example, fish, birds, worms and insects. In some embodiments, a subject is a human.

As used herein, “biological activity” means the ability of a compound (e.g., a drug) to produce a desired result. Such a determination will vary based on the compound of interest and its intended use and desired result. For example, the biological activity may be determined based on the ability of the particular drug (e.g., the methylsulfinyl or methylsulfide form of a drug) to produce a therapeutically beneficial effect, such as ameliorating existing symptoms, preventing additional symptoms, ameliorating or preventing the underlying metabolic causes of symptoms, postponing or preventing the further development of a disorder and/or reducing the severity of symptoms that will or are expected to develop. In other embodiments, the biological activity may be determined based on the ability of the compound (e.g., the methylsulfinyl or methylsulfide form of the compound) to act as a pesticide (e.g., insecticide and rodenticide). Determinations of “higher biological activity” will be made through comparisons of the activity of a compound in its methylsulfinyl-oxidized form compared to the activity of the methylsulfide-reduced form of the same compound.

Biological activity may be determined using appropriate assays depending on the compound and the anticipated effect. One of skill in the art will be able to determine which assay is appropriate under each particular set of circumstances. Assays may be conducted in vivo or in vitro. For example, an assay may be a [3H]S-piperone binding assay to rabbit striatal DA (Dopamine) receptors, competing with THD (Thioridazine), MES (Mesoridazine), and SUL (Sulforidazine) (see Niedzwiecki, D. M., et al. (1989) The Journal of Pharmacology and Experimental Therapeutics, 250(1):117-125); or an in vivo assay to detect injected sulmazole and its metabolite using a high-performance liquid chromatographic assay with fluorescence detection for sulmazole and its metabolites (see Roth, W. (1983) Journal of Chromatography, 278(2): 347-357).

A compound for use in the methods described herein may be any compound that contains a methylsulfinyl moiety. In some embodiments, the compound is a drug having a methylsulfinyl moiety. For example, the drug can be chosen from enoximone; pergolide; lincomycin; thiethylperazine; fensulfothion; nifuratel; albendazole; modafinil; captodiame; sulfinpyrazone; clindamycin; thiocolchicoside; omeprazole; flosequinan; dimethylsulfoxide; sulmazole; triclabendazole; mesoridazine; oxisuran; and sulindac. The methods described herein will apply equally well to compounds known at the time of filing and those to be created in the future.

An excess amount of an epimer, as used herein, refers to any amount that results in greater than about 50% by weight (e.g., greater than about 55% by weight; greater than about 60% by weight; greater than about 65% by weight; greater than about 70% by weight; greater than about 75% by weight; greater than about 80% by weight; greater than about 85% by weight; greater than about 90% by weight; greater than about 92% by weight; greater than about 95% by weight; greater than about 97% by weight; greater than about 98% by weight; and greater than about 99% by weight) of the desired epimer relative to the undesired epimer. For example, when an excess amount of the R-epimer is desired, the R-epimer can be present in an amount of at least 75% by weight compared to the S-epimer. In some embodiments, the R-epimer can be present in an amount of at least 90% by weight compared to the S-epimer. When an excess amount of the S-epimer is desired, on the other hand, the S-epimer can be present in an amount of at least 75% by weight compared to the R-epimer. In some embodiments, the S-epimer can be present in an amount of at least 90% by weight compared to the R-epimer.

Epimers can be separated by methods known in the art, for example, epimers can be separated using chiral HPLC and chiral GC methods. Alternatively, the particular epimers can be directly synthesized using methods known in the art. For example, see Choi, S., Haggart, D., Toll, L., & Cuny, G. D. (2004) Bioorganic & Medicinal Chemistry Letters, 14(17): 4379-4382; Berthod, A., Xiao, T. L., Liu, Y., McCulla, R. D., Jenks, W. S., & Armstrong, D. W. (2002) Journal of Chromatography A, 955(1):53-69; del Nozal, M. J., Toribio, L., Bernal, J. L., Nieto, E. M., & Jimenez, J. J. (2002) Journal of Biochemical and Biophysical Methods, 54(1-3):339-345; Cass, Q. B., & Batigalhia, F. (2003) Journal of Chromatography.A, 987(1-2), 445-452; and El Ouazzani, H., Khiar, N., Fernandez, I., & Alcudia, F. (1997) The Journal of Organic Chemistry, 62(2), 287-291.

In some embodiments, the compound may be a prodrug having a methylsulfinyl moiety. Further provided herein is a method for treating a subject with a prodrug comprising a methylsulfinyl moiety. The method can include determining whether the drug comprising the methylsulfinyl moiety exhibits higher biological activity when the methylsulfinyl moiety is present in the methylsulfinyl-oxidized form or the methylsulfide-reduced form; and administering, to the subject, a composition comprising the prodrug in an excess amount of the R-epimer relative to the S-epimer if the methylsufinyl-oxidized form of the drug exhibits higher biological activity, or a composition comprising the prodrug in an excess amount of the S-epimer relative to the R-epimer if the methylsulfide-reduced form of the drug exhibits higher biological activity.

As used herein, “administration” refers to delivery of a compound having a methylsulfinyl moiety by any external route, including, without limitation, IV, intramuscular, SC, intranasal, inhalation, transdermal, oral, rectal, sublingual, and parenteral administration. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions. Dosages may also be modified based on the increased activity which may be exhibited when the amount of the active or higher activity epimer is increased compared to the amount of inactive or lower activity epimer. One of skill in the art would be able to make such modifications based, for example, the results of activity assay and the amount of the two epimers present.

The compounds can be provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. Actual methods of preparing such dosage forms are known, or will be apparent, to those skilled in this art; for example, see Remington's Pharmaceutical Sciences, Mack Publishing Company, Easton, Pa., 15th Edition, 1975.

Also provided herein is a method for treating a subject with a compound comprising a methylsulfinyl moiety. The method can include determining whether the compound comprising the methylsulfinyl moiety exhibits higher biological activity when the methylsulfinyl moiety is present in the methylsulfinyl-oxidized form or the methylsulfide-reduced form; and contacting the subject with a composition comprising the compound in an excess amount of the R-epimer relative to the S-epimer if the methylsulfinyl-oxidized form exhibits higher biological activity, or a composition comprising the compound in an excess amount of the S-epimer relative to the R-epimer if the methylsulfide-reduced form exhibits higher biological activity. In some embodiments, the biological activity is insecticidal activity.

The term “contacting” means bringing together a subject and a composition comprising one or more compounds having a methylsulfinyl moiety. In some embodiments, contacting occurs through external application of the composition. In some embodiments, the external application is followed by adsorption of the compound into the subject. In some embodiments, contacting occurs through administration or ingestion of the composition.

Further provided herein is a method for increasing the shelf-stability of a compound (e.g., a drug) comprising a methylsulfinyl moiety. The method can include determining whether the compound comprising the methylsulfinyl moiety exhibits higher biological activity when the methylsulfinyl moiety is present in the methylsulfinyl-oxidized form or the methylsulfide-reduced form; and formulating either 1) a composition comprising an oxidant and the compound in an excess amount of the R-epimer relative to the S-epimer if the methylsulfinyl-oxidized form exhibits higher biological activity, or 2) a composition comprising a reductant and the compound in an excess amount of the S-epimer relative to the R-epimer if the methylsulfide-reduced form exhibits higher biological activity. In some embodiments, the compound is a drug having a methylsulfinyl moiety.

As used herein, an oxidant can be any chemical compound that readily transfers oxygen atoms, or any substance that gains electrons in a redox chemical reaction. In some embodiments, the oxidant is a pharmaceutically acceptable oxidant, i.e., an oxidant that is non-toxic and does not cause undesired side reactions. Those skilled in the art will be able to select and identify suitable oxidants for use in the methods described herein by routine tests of known non-toxic mild oxidants. For example, the oxidant can be hydrogen peroxide, hypochlorous acid, urea peroxide, sodium perborate tetrahydrate, sodium percarbonate, sodium perborate, sodium peroxide, sodium periodate, calcium peroxide, or mixtures thereof. In some embodiments, the oxidant is hydrogen peroxide, hypochlorous acid, or mixtures thereof.

As used herein, a reductant can be any chemical compound that is the element or compound in a redox reaction that reduces another species (i.e., donates the electron(s)). In some embodiments, the reductant is a pharmaceutically acceptable reductant. Those skilled in the art will be able to select and identify suitable reducants for use in the methods described herein by routine tests of known non-toxic mild reductants. For example, the reducant can be dithiothreitol (DTT), a thioredoxin, sodium dithionite, sodium bisulphite, ascorbic acid, sodium ascorbate, calcium ascorbate, palmityl-DL-ascorbic acid, propyl gallate, octyl gallate, dodecyl gallate, butylhydroxyanisole (BHA) gallate and butylhydroxytoluene (BHT) gallate, formamidine sulphinic acid, stannous ion (e.g., stannous chloride or stannous tartrate), Fe(II), Cu(I), erythrobate, α-tocopherol, γ-tocopherol, δ-tocopherol, oxalic acid, formic acid, or mixtures thereof.

This disclosure also provides a method for increasing the in vivo activity of a drug comprising a methylsulfinyl moiety in a subject. The method can include determining whether the drug comprising the methylsulfinyl moiety exhibits higher biological activity when the methylsulfinyl moiety is present in the methylsulfinyl-oxidized form or the methylsulfide-reduced form; and administering, to the subject, either 1) an oxidant and a composition comprising the drug in an excess amount of the R-epimer relative to the S-epimer if the oxidized form exhibits higher biological activity; or 2) a reductant and a composition comprising an excess amount of the S-epimer relative to the R-epimer if the reduced form exhibits higher biological activity.

In some embodiments, the oxidant or the reductant is administered before or after the drug. In some embodiments, the oxidant or the reductant and the drug are administered together. In some embodiments, the oxidant or reductant is formulated into the composition comprising the drug.

This disclosure further provides compositions. In some embodiments, the composition is prepared for a drug having a methylsulfinyl moiety wherein the drug exhibits higher biological activity when the methylsulfinyl moiety is present in the methylsulfinyl-oxidized form. In such cases, the composition can include an oxidant and the drug in an excess amount of the R-epimer relative to the S-epimer. In some embodiments, the composition is prepared for a drug having a methylsulfinyl moiety wherein the drug exhibits higher biological activity when the methylsulfinyl moiety is present in the methylsulfide-reduced form. In such cases, the composition can include a reductant and the drug in an excess amount of the S-epimer relative to the R-epimer.

The compositions disclosed herein can further include a pharmaceutically acceptable carrier or diluent. Pharmaceutical carriers and diluents suitable for administration of the compounds provided herein include any such carriers known to those skilled in the art to be suitable for the particular mode of administration. Pharmaceutically acceptable carriers and diluents include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of compounds of the formulae described herein. In some embodiments, the carrier or diluent is a physiologically acceptable saline solution. In some embodiments, the carrier or diluent is dimethylsulfoxide (DMSO).

Certain pharmaceutically acceptable carriers or diluents contain a methylsulfinyl moiety (e.g., DMSO). In some embodiments, when a drug having a methylsulfinyl moiety exhibits higher biological activity when the methylsulfinyl moiety is present in the methylsulfinyl-oxidized form, the drug can be administered or formulated with a pharmaceutically acceptable carrier or diluent having a methylsulfinyl moiety. In some embodiments, such a combination could inhibit or reduce the rate of reduction of the methylsulfinyl moiety of the drug and therefore increase the efficacy of the drug. Without being bound by theory, such a result can be linked to the excess concentration of the carrier, and thus the methylsulfinyl moiety on the carrier, compared to that of the drug. The carrier can inhibit the reduction of the drug by MsrA by competing for available enzyme. In some embodiments, the drug is present having an excess amount of the R-epimer relative to the S-epimer.

Alternatively, when a drug having a methylsulfinyl moiety exhibits higher biological activity when the methylsulfinyl moiety is present in the methylsulfide-reduced form, the drug can be administered or formulated with a pharmaceutically acceptable carrier or diluent lacking a methylsulfinyl moiety. In some embodiments, such a combination will avoid inhibition of the reduction of the methylsulfinyl moiety of the drug and therefore increase the efficacy of the drug. Without being bound by theory, such a result can be linked to the lack of a methylsulfinyl moiety on the carrier which could compete with that of the drug. In some embodiments, the drug is present having an excess amount of the S-epimer relative to the R-epimer.

Also provided herein is a method of increasing the amount of a particular epimer of a compound having a methylsulfinyl moiety in a composition. In one embodiment, the method includes treating a mixture of epimers (e.g., through a column) with the appropriate Msr enzyme to reduce one of the epimers to the methylsulfide-reduced form, thereby increasing the amount of the other epimer of interest. For example, a mixture of epimers could be contacted with MsrA to reduce the S-epimer to the corresponding methylsulfide. The resulting methylsulfide then could be removed from the sample using techniques known in the art (e.g., chromatography and crystallography), thereby enriching for the R-epimer. Alternatively, a mixture of epimers could be contacted with fRMsr to reduce the R-epimer to the corresponding methylsulfide, thereby enriching for the S-epimer. As those of skill would understand, one could remove the remaining R- or S-epimer, depending upon which enzyme was used, and enrich for the methylsulfide-reduced form. Without being bound by theory, such use could reduce the reliance on the MsrA enzymes present in vivo to reduce the methylsulfinyl moiety to the methylsulfide, and accordingly result in a higher concentration of the active compound in the subject. In some embodiments, such techniques can be utilized with natural compounds having methylsulfinyl moieties (e.g., sulforaphane from broccoli) or therapeutic drugs.

Further provided herein is a method of modifying the rate of reduction of a compound having a methylsulfoxide group. The method can include replacing a sulfoxide moiety within the compound with a methylsulfinyl moiety. For example, diethylsulfoxide is resistant to reduction by Msrs enzymes, however, replacement of one ethyl group with a methyl group could make the compound a substrate for MsrA (e.g., if the resulting compound is methylethyl-S-sulfoxide) or could make it resistant to reduction (e.g., if the resulting compound is methylethyl-R-sulfoxide). Without being bound by theory, such modifications could reduce or accelerate the rate of reduction of the compound based on the modification made to the substituents and/or stereochemistry of the compound.

### EXAMPLES

### Example 1

### General Methods

Preparation of free methionine-S-sulfoxide (Met-SO) and methionine-R-sulfoxide (Met-RO)—Free Met-SO and Met-RO were prepared from a mixture of L-Met-R,S-sulfoxide (Met-RSO; Sigma) according to the method of Lavine (Lavine, T. F. (1947) J. Biol. Chem. 169, 477-491). To obtain diastereomers of higher purity, the separation process was repeated twice for each sulfoxide. Purity of Met-SO and Met-RO was assessed by an HPLC procedure using o-phthalaldehyde (OPA) (Sigma)-derivatized amino acids (Sharov, V. S., Ferrington, D. A., Squier, T. C., Schöneich, C. (1999) FEBS Lett. 455, 247-250) and found to exceed 98%.

Cell culture—SK-Hep1 (ATCC: HTB-52™) and fRMsr-transfected SK-Hep1 cells were cultured in DMEM or Met-free DMEM (GIBCO) media, supplemented with 0.1 mM Met, 0.1 mM Met-RO, 0.1 mM Met-SO, or 0.1 mM Met-RSO. The media also contained 10% dialyzed fetal bovine serum (FBS) and an antibiotics-antimycotic mixture (GIBCO) of 100 units/mL penicillin G sodium, 100 μg/mL streptomycin sulfate, and 0.25 μg/mL amphotericin B. In initial experiments, the media containing different amounts of Met was examined and it was found that 0.1 mM Met was optimal and avoided Met deficiency. Thus, 0.1 mM Met sulfoxides were used in the experiments described below. Cell culture experiments, with the exception of a proliferation assay, were carried out in 6-well plates, and cells were maintained at 37° C. in a 5% CO₂ atmosphere.

Growth of SK-Hep1 cells was analyzed in modified DMEM media containing Met, Met-SO, Met-RO, or Met-RSO (0.1 mM of each amino acid) or with no addition of these compounds. In addition, media were supplemented or not with 100 nM sodium selenite. Cells were analyzed at 0, 24, 48, and 72 hours. Another experiment involving SK-Hep1 cells was performed with cells grown in serum-free modified DMEM medium, containing insulin (5 μg/mL) and transferrin (10 μg/mL), Met or Met-RSO (0.1 mM of each amino acid) and 100 nM sodium selenite (or with no addition of this compound), and separately in 10% dialyzed FBS-containing DMEM medium containing 0.1 mM Met with 100 nM sodium selenite (or not). Cells were analyzed at 0, 24, 48, and 72 hours. Cell growth assays were carried out as described below.

Proliferation Assay—Cell growth was quantified using colorimetric MTS assay (Promega). Cells in regular DMEM medium were plated in 96-well plates at 5×10³ cells/well, washed with PBS, and specialized DMEM media were added that contained Met, Met-SO, Met-RO, or Met-RSO 24 hours after plating. To assay for cell proliferation, a tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine methosulfate; PMS) were mixed according to the manufacturer's protocol and then 20 μL of the mixture were added to 100 μL of phenol red-excluded media that further replaced cell culture media. After 90 minutes of incubation at 37° C. in the atmosphere of 5% CO₂, cell proliferation was measured at indicated time periods from 0 to 96 hours at 450 nm in a plate reader. Direct counting of viable cells using 0.2% trypan blue was done in 6-well plates.

HPLC analysis of media samples—Cells were plated in 6-well plates at 7.5×10⁴ cells/well in 1.5 mL of regular DMEM medium. After 24 hours, cells were washed with PBS and the medium was changed to a modified DMEM containing Met, Met-SO, Met-RO, or Met-RSO (0.1 mM of each amino acid). 90 μL of medium from each well was collected at 0, 24, 48, 72, 96 hours and mixed with 10 μL of 100% TCA. After incubation at 4° C. for 10 minutes and centrifugation at 13,000 rpm for 15 minutes, the supernatant (50 μL) was diluted 10 times with distilled water and prepared for OPA derivatization. OPA derivatization of amino acids and HPLC analysis was performed as described (Sharov, V. S., Ferrington, D. A., Squier, T. C., Schöneich, C. (1999) FEBS Lett. 455, 247-250) with minor modifications. The derivatization reagent was freshly prepared as a stock solution (40 mg of o-phthalaldehyde, 1 mL of methanol, 50 μL, of 2-mercaptoehanol, 5 mL of 0.1 M Na₂B₄O₇, pH 9.5) at room temperature in a capped amber vial. Sample solutions (2-5 μL) were mixed with the OPA derivatization reagent to 100 μL, final volume. Following a 2 minute reaction at room temperature, the mixture was fractionated on a Zorbax Eclipse XDB-C8 column (4.6×150 mm). Changes in Met levels in the fRMsr activity assay were similarly quantified. Detection was by fluorescence of Met derivatives using a Waters 474 scanning fluorescence detector with excitation at 330 nm and emission at 445 nm.

Knockdown of MsrA in SK-Hep1 cells by siRNA—Double stranded siRNAs (Dharmacon) for targeting human MsrA mRNA were used to knockdown this gene in SK-Hep1 cells. Cells were transfected with siRNA by using DharmaFECT™ Transfection Reagent (Dharmacon) according to the manufacturer's protocol. Proliferation of these cells was assayed 60 hours after transfection in comparison with control transfected cells.

Cloning and expression of yeast fRMsr—Characterization of S. cerevisiae fRMsr (YKL069W) is described in Le, D. T., et al. (2009) Functional analysis of free methionine-R-sulfoxide reductase from Saccharomyces cerevisiae. J Biol Chem., 284(7), 4354-4364. Briefly, a yeast fRMsr cDNA, which encodes a protein homologous to fRMsr from E. coli, was amplified from S. cerevisiae genomic DNA and subcloned into pET21b vector (Novagen) using primers 5′-AAA CAT ATG ATG GGC TCA TCA ACC GGG TTT C-3′ (sense; SEQ ID NO:1) and 5′-AAG CGG CCG CGA CAC ATG ATT TAT TAA TTA ATT TAG CAA G-3′ (antisense; SEQ ID NO:2). After transformation into BL21 (DE3) E. coli, the subcloned sequence was verified by DNA sequencing. Cells with the plasmid in 500 mL LB medium containing 100 μg/mL ampicillin were grown until OD₆₀₀ reached 0.6-0.8, followed by addition of IPTG to 0.3 mM. Protein expression was at 30° C. for 4 hours, followed by harvesting cells by centrifugation at 4,000 rpm for 5 minutes. Cells were washed with PBS and stored at −70° C. until use.

To purify the protein, a cell pellet was dissolved in resuspension buffer (Tris-HCl, pH 7.5, 15 mM imidazole, 300 mM NaCl), and PMSF was added to a final concentration of 0.5 mM. After sonication, the supernatant was collected by centrifugation at 8,000 rpm for 30 minutes. The supernatant was loaded onto a cobalt Talon resin (Clontech) pre-equilibrated with resuspension buffer. Following washing with the same buffer, the protein was eluted with elution buffer (Tris-HCl, pH 7.5, 300 mM imidazole, 300 mM NaCl). Fractions containing yeast fRMsr were pooled together and dialyzed overnight against PBS in a dialysis cassette (Pierce).

Transfection of yeast fRMsr gene into SK-Hep1 cells—A 6 His-tag sequence was cloned at the C-terminus of yeast fRMsr, the sequence was inserted into a mammalian expression vector, pCI-neo (Promega), and the resulting construct was verified by DNA sequencing. SK-Hep1 cells were transfected or cotransfected with the expression vector coding for fRMsr and pEGFP-N1 (Clontech) vector or with an empty pCI-neo vector and pEGFP-N1 vector using FuGENE 6 Transfection Reagent according to the manufacturer's suggestion. To establish a stable cell line, cells were selected in the presence of 800 μg/mL G418 sulfate (Promega), and further maintained in the presence of 400 μg/mL G418 sulfate. Expression of recombinant fRMsr was verified by western blotting using anti-His-tag antibodies and Msr activities were assayed by an HPLC method using OPA derivatization.

Activity assays of fRMsr—Reaction mixture (40 μL) included 50 mM DTT, 1 mM substrate (Met-RO or Met-SO), and purified enzyme or cell lysate prepared by treatment with CelLytic™M cell lysis reagent (Sigma). 20 μL of the reaction mixture was mixed with 2 μL of TCA and subjected to OPA derivatization as described above and then injected onto the column to measure endogenous Met level in the samples. An additional 20 μL from the same original reaction mixture were incubated at 37° C. for 30 minutes and then subjected to OPA derivatization by the same procedure using TCA. After derivatization of the sample (2-5 μL) by adding OPA solution to 100 μL as a final volume, 50 μL of the mixture were subjected to an HPLC separation as described above.

Analysis of SK-Hep1 and fRMsr-transfected SK-Hep1 cells—SK-Hep1 and fRMsr-transfected SK-Hep1 cells were plated in 6-well plates at 7.5×10⁴ cells/well in regular DMEM medium. The medium was changed to Met-free DMEM supplemented with either Met or individual Met sulfoxides at 24 hours after plating. Cells were then collected every 24 hours until 96 hours. Protein concentration was measured by the Bradford assay and the samples were probed by standard immunoblot assays with polyclonal MsrA (kindly provided by Bertrand Friguet), cystathionine beta synthase (CBS) (kindly provided by Ruma Banerjee), and activating transcription factor 3 (ATF3) (Santa Cruz Biotechnology) antibodies.

Microscopy—A Nikon TE-300 Widefield Microscope was used to prepare images of SK-Hep1 cells grown on different Met and Met sulfoxide media. Microscopy analyses were carried out in the University of Nebraska Microscopy Core Facility.

⁷⁵ Se Metabolic labeling—SK-Hep1 cells were plated in 6-well plates at 7.5×10⁴ cells/well in 1.5 mL of regular DMEM medium. After 24 hours, cells were washed with PBS and the medium was changed to modified DMEM media containing Met, Met-SO, Met-RO, or Met-RSO (0.1 mM of each amino acid). 0.01 mCi of freshly neutralized [⁷⁵Se]selenious acid (specific activity 1,000 Ci/mmol, University of Missouri Research Reactor) was added in each well at 48 hours after changing the medium and cells were incubated at 37° C. for 24 hours in 5% CO₂ atmosphere, followed by harvesting the cells. Cell extracts (30 μg of total protein) were electrophoresed on 10% Bis-Tris gels and transferred onto PVDF membranes (Invitrogen). The ⁷⁵Se radioactivity pattern was visualized by using a PhosphorImager (GE Health Care).

Resistance of fRMsr-expressing and control SK-Hep1 cells to oxidative stress—SK-Hep1 cells stably transfected with either an empty pCI-neo vector and fRMsr construct were plated in 96-well plates at 3.0×10⁴ cells/well. The medium was changed to a serum-free medium and H₂O₂ was added to each well at indicated concentrations (0-1000 μM). Cell viability was measured using colorimetric MTS assay.

Mice—MsrA knockout mice (Moskovitz, J., Bar-Noy, S., Williams, W. M., Requena, J., Berlett, B. S., Stadtman, E. R. (2001) Proc. Natl. Acad. Sci. USA 98, 12920-12925) were used. These and control C57BL6 mice were used for blood sampling. In addition, C57BL6 mice, which were subjected to selenium deficiency or a control diet containing 0.4 ppm Se in the form of sodium selenite for 8 months, were used.

HPLC analysis of Met-RO and Met-SO level in mouse blood—Mouse blood was centrifuged at 13,000 rpm for 15 minutes. The supernatant was prepared for OPA derivatization without dilution following TCA precipitation and analyzed at isocratic flow rate of 2 min/mL at 89:11 (v/v) of 20 mM sodium acetate, pH 5.8 (solvent A) and methanol (solvent B).

### Example 2

### SK-Hep1 Cells Grow in the Presence of Met and Met-SO, but not in the Presence of Met-RO

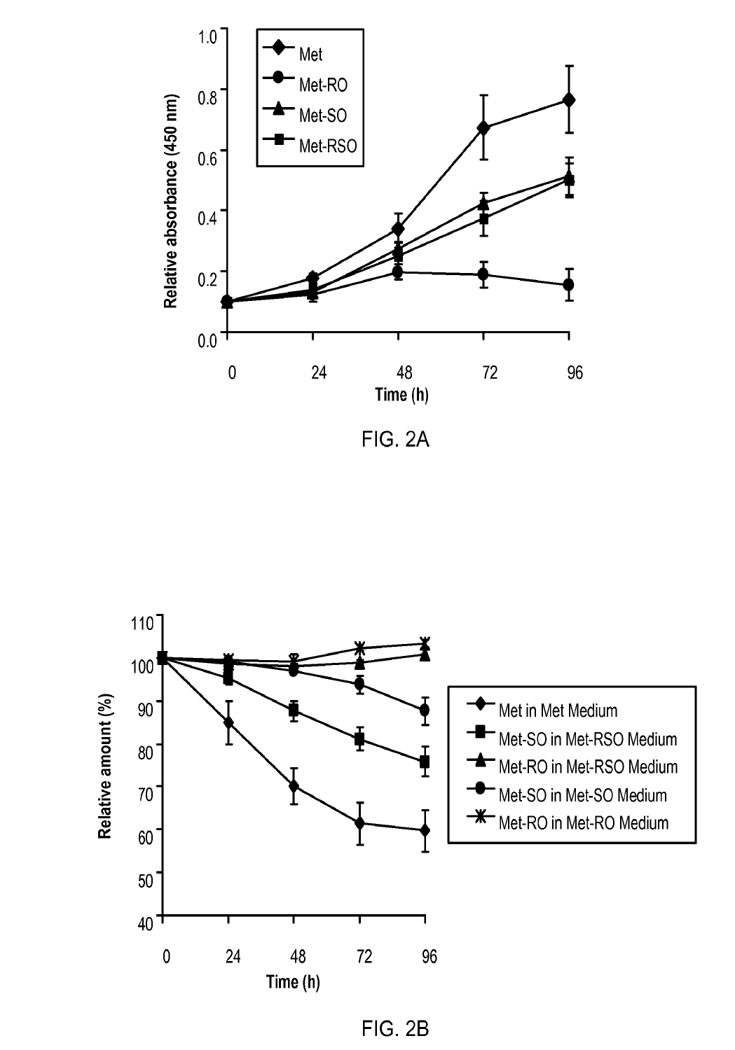

To examine the capacity of mammalian cells to provide Met for cellular metabolism by Met sulfoxide reduction, human hepatoma SK-Hep1 cells were grown in Met-free DMEM medium supplemented with 0.1 mM Met, 0.1 mM Met-SO, 0.1 mM Met-RO or 0.1 mM mixed Met-RO/Met-SO (Met-RSO) (FIG. 2A; Met (diamonds), Met-RO (triangles), Met-SO (circles), and Met-RSO (squares)). SK-Hep1 hepatocytes grew best in the presence of Met and could also proliferate in Met-SO and Met-RSO media, although at a reduced rate. In contrast, Met-RO did not support growth of SK-Hep1 cells. Morphology of cells maintained on Met-SO and Met-RO media was also different (see below). After 96 hours on the Met-RO medium, SK-Hep1 hepatocytes were long and narrow, similar in shape to Met-restricted cells, whereas the cells grown on Met-SO resembled those maintained in the presence of Met. These data suggested that SK-Hep1 cells have a system for import and reduction of Met-SO, which provides them with Met, whereas these cells were unable to utilize free Met-RO.

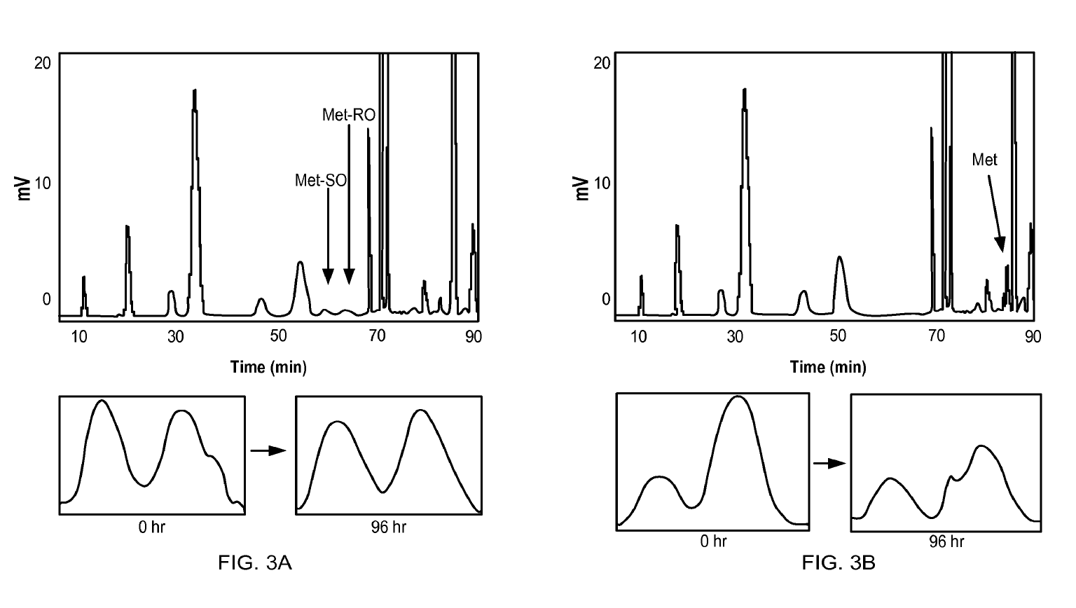

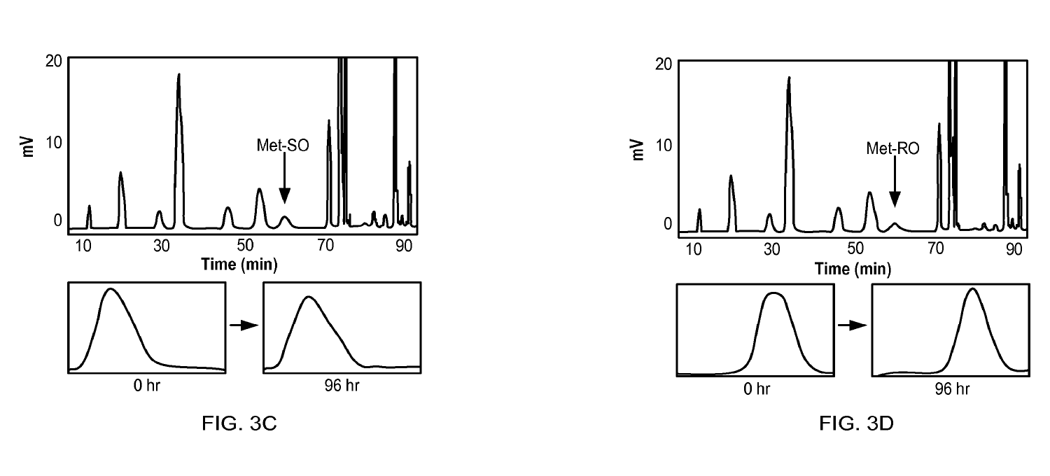

To determine whether Met, Met-RO, and Met-SO were consumed by SK-Hep1 hepatocytes, the levels of these compounds were determined in each growth medium at 0, 24, 48, 72, and 96 hours. For this purpose, an HPLC procedure was developed that utilized OPA derivatization of free amino acids following TCA precipitation of medium components (FIGS. 2B and 3). It was determined that the relative amounts of Met in the Met-supplemented medium decreased to approximately 60% of the initial level at 96 hours. In contrast, Met-RO levels were not changed in Met-RO and Met-RSO media at any time points. Met-SO was decreased to 88% in the Met-SO-supplemented medium and to 77% in the Met-RSO medium (FIG. 2B). These data indicate that SK-Hep1 cells could import and consume both Met-SO and Met from the medium to support cell proliferation. On the other hand, consistent with the inability of SK-Hep1 cells to grow on Met-RO, this compound was not utilized. Moreover, when cells were grown on Met-RSO, only Met-SO was consumed.

### Example 3

### SK-Hep1 Cell Extracts are Active in the Reduction of Met-SO but do not Reduce Met-RO

Since both MsrA and MsrB were reported to support low level reduction of free Met sulfoxides in in vitro assays (Kim, H. Y., Gladyshev, V. N. (2004) Mol. Biol. Cell. 15, 1055-1064), the contribution of these enzymes to Met sulfoxide reduction under physiological conditions in mammalian cells was examined. The specific activities of SK-Hep1 cell extracts were measured for the reduction of free Met-RO and Met-SO. No free Met-RO reductase activity was detected (it was within background), while the activity for free Met-SO reduction was ≈38.6 pmol/min/mg protein (Table 1).

TABLE 1
Specific activities of SK-Hep1 cells towards free Met sulfoxides.
Specific activity for each Met-O (pmol/min/mg)

| | Met-RO | Met-SO |
|---|---|---|
| **yfRMsr | 30015 ± 2652 | ND* |
| SK-Hep1 | ND* | 38.6 ± 3.6 |
| yfRMsr transfected SK-Hep1 | 46.4 ± 5.0 | 42.7 ± 5.9 |

*A signal corresponding to specific activity less than 5 pmol/min/mg is shown as ND (Not Detectable)
**yfRMsr is yeast free methionine R-sulfoxide reductase
Table shows values ± standard deviations from 3 independent experiments

### Example 4

### Role of MsrA in the Reduction of Free Met-SO

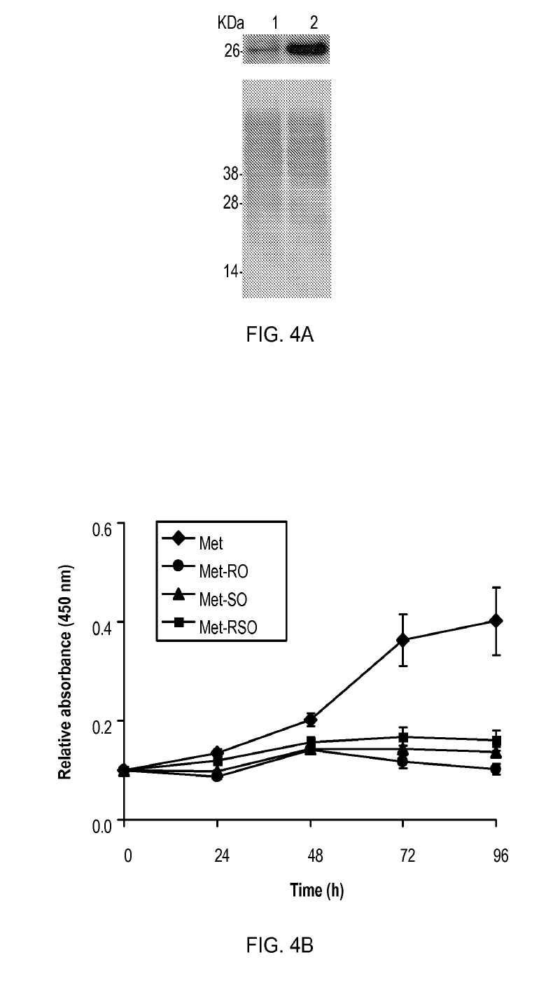

To examine the role of MsrA in providing cells with Met by reduction of free Met-SO, its expression by siRNA was knocked-down in SK-Hep1 cells. Decreased MsrA expression was verified by Western blot assays (FIG. 4A). As shown in FIG. 4B, MsrA-knockdown SK-Hep1 cells were grown in media containing 0.1 mM Met (closed diamonds), Met-RO (closed circles), Met-SO (closed triangles), or Met-RSO (closed squares) for 96 hours. Cell growth was measured by an MTS cell proliferation assay at 0, 24, 48, 72, and 96 hours. Error bars represent standard deviations from 3 independent experiments. It was found that MsrA-deficient cells grew neither in Met-SO nor Met-RO media, whereas Met still supported their growth. Thus, MsrA is responsible for the reduction of Met-SO acquired from the media by SK-Hep1 cells. These data also indicated that none of the three mammalian MsrB isozymes contributes significantly to the enzymatic reduction of free Met sulfoxides or to providing Met to support cell growth.

### Example 5

### A Yeast Enzyme Specific for Free Met-RO

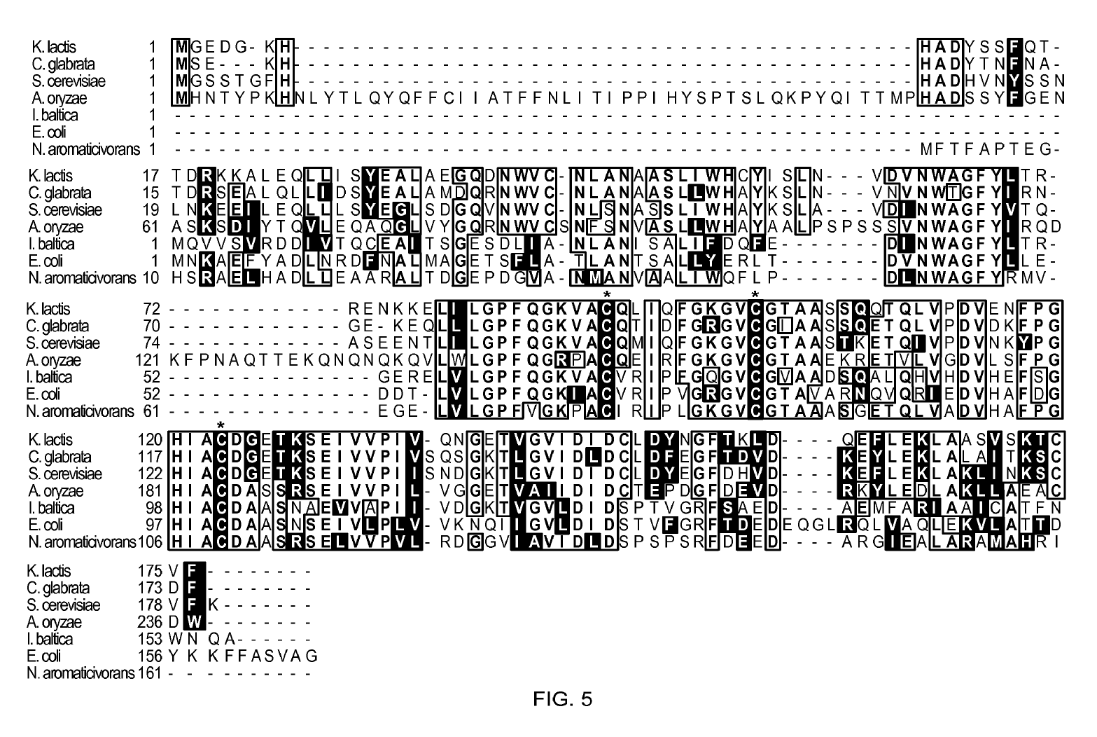

A recent study identified a bacterial enzyme specific for free Met-RO (Etienne, F., Spector, D., Brot, N., Weissbach, H. (2003) Biochem. Biophys. Res. Commun. 300, 378-382). This GAF domain-containing protein was designated as fRMsr. However, homologs of this protein were detected in many lower eukaryotes (Le D. T., et al., (2009) J Biol Chem., 284(7), 4354-4364). Multiple sequence alignment of yeast fRMsr and its orthologs in bacteria, yeast, algae, and fungi are shown in FIG. 5, and the Cys residues involved in catalysis are highlighted for accession numbers: S. cerevisiae (NP_{—}012854), K. lactis (XP_{—}456263), C. glabrata (XP_{—}446236), A. oryzae (BAE62132), N. aromaticivorans DSM 12444 (YP_{—}495413), I. baltica OS145 (ZP_{—}01043850), and E. coli F11 (ZP_{—}00723710).

A fRMsr homolog from S. cerevisiae was cloned and expressed in E. coli as a His-tagged protein as described herein. The recombinant protein was soluble and had the expected molecular weight as determined by SDS-PAGE and mass spectrometry, and functioned in a manner similar to the native yeast protein fRMsr in S. cerevisiae cells (Le D. T., et al., (2009) J Biol Chem., 284(7), 4354-4364). The recombinant yeast fRMsr protein exhibited high activity towards free Met-RO (≈33 nmol/min/mg protein), whereas it was inactive with Met-SO as well as with dabsyl-Met-RO and dabsyl-Met-SO.

### Example 6

### Yeast fRMsr-expressing SK-Hep1 Cells Grow on Met-RO

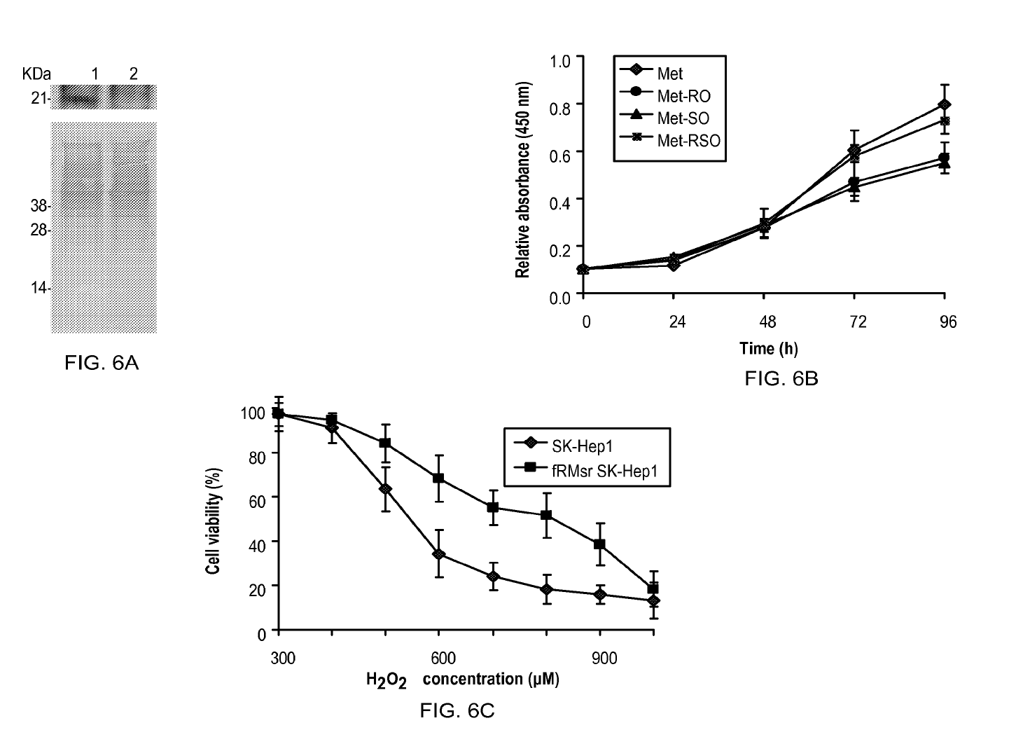

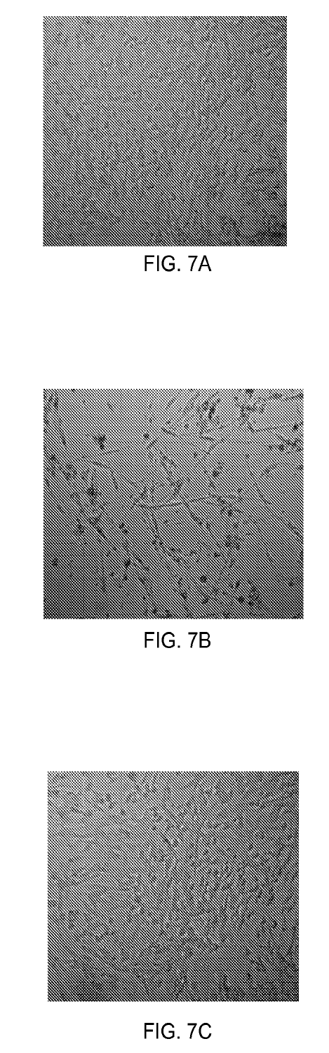

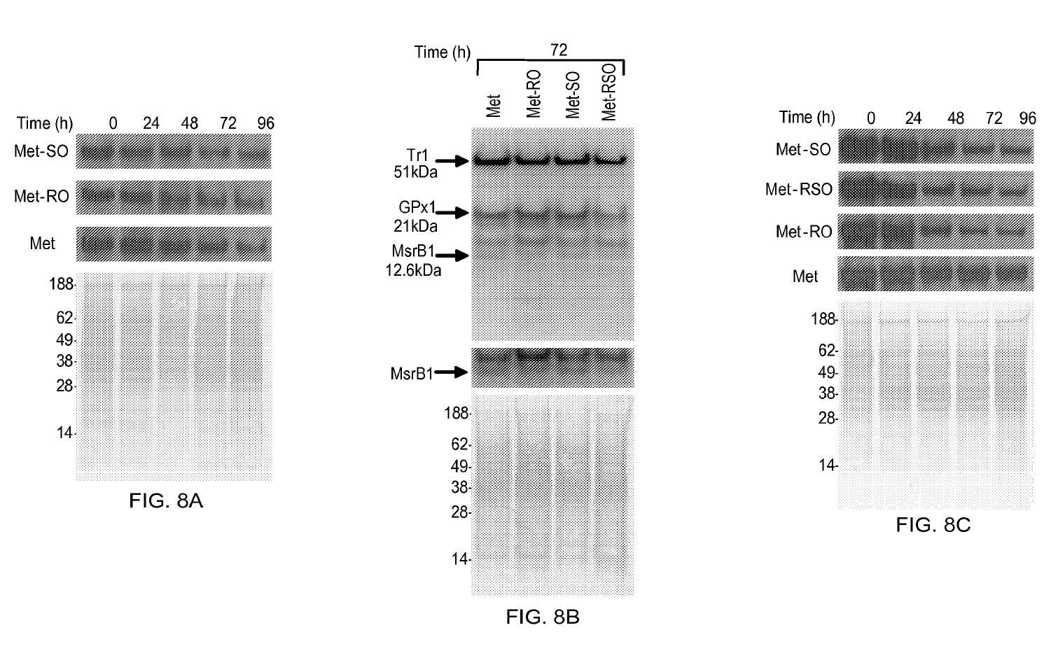

Yeast fRMsr was stably expressed in SK-Hep1 cells in the form of a His-tagged tagged protein, and its expression was verified in Western blot assays with antibodies specific for the His-tag (FIG. 6A). Specific activity of fRMsr-transfected cells for Met-RO was ≈46.4 pmol/min/mg protein and the activity of these cells towards Met-SO was ≈42.7 pmol/min/mg protein (Table 1). The transfected cells grew on Met-SO or Met-RO media, and their growth was essentially indistinguishable from that of cells grown in the presence of Met (FIG. 6B). Morphology of fRMsr expressing cells on the Met-RO-supplemented medium was similar to that of cells grown on Met (FIG. 7). Thus, yeast fRMsr expressed in SK-Hep1 cells could reduce free Met-RO in quantities sufficient to compensate for Met deficiency. These data also indicated that Met-RO could be imported into SK-Hep1 cells from the medium.

### Example 7

### Increased Resistance of fRMsr-expressing SK-Hep1 Cells to Oxidative Stress

SK-Hep1 cells expressing yeast fRMsr were examined for resistance to oxidative stress by subjecting them (and control cells) to hydrogen peroxide treatment. At higher concentrations of hydrogen peroxide (above 400 μM), fRMsr-expressing cells showed significantly higher viability than control cells (FIG. 6C). Thus, yeast fRMsr protected SK-Hep1 cells from oxidative stress caused by hydrogen peroxide treatment. The increased resistance of transfected cells to oxidative stress is likely due to reduction of free Met-RO formed by Met oxidation in the presence of hydrogen peroxide. In addition, these data indicate that reversible oxidation and reduction of free Met-RO (and by analogy free Met-SO) provides mammalian cells with an antioxidant defense system.

### Example 8

### Expression of MsrA, Selenoprotein, CBS, and ATF3 in Cells Grown on Met and Met Sulfoxide Media

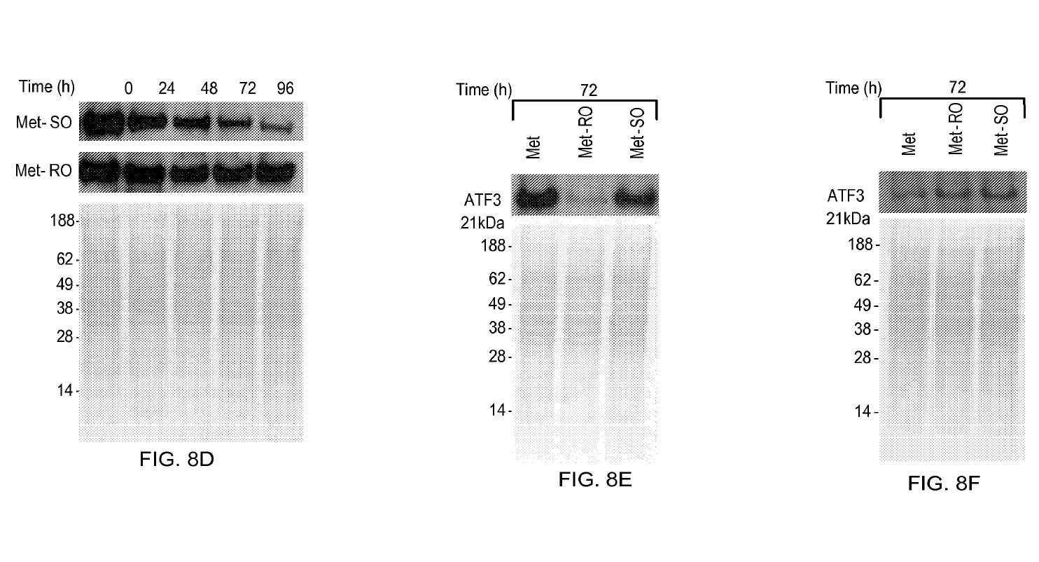

Regulation of expression of MsrA by availability of Met or Met sulfoxides was examined by maintaining cells in the corresponding growth media (FIG. 8). FIG. 8A provides a Western blot analysis of MsrA in SK-Hep1 cells grown in media containing 0.1 mM Met, Met-RO, or Met-SO at 0, 24, 48, 72, and 96 hours. FIG. 8B details the metabolic labeling of SK-Hep1 cells grown in media containing Met, Met-RO, Met-SO, or Met-RSO, with ⁷⁵Se. Cell extracts were analyzed by SDS-PAGE and the ⁷⁵Se pattern visualized with a PhosphorImager (upper panel). Migration of major selenoproteins, thioredoxin reductase1 (Tr1) and glutaredoxin peroxidase1 (GP×1) is indicated. Migration of MsrB1 is also shown, and the corresponding area of the gel is enlarged in the middle panel. Lower panel shows protein loading. FIG. 8C illustrates a Western blot analysis of CBS in SK-Hep1 cells grown in media with 0.1 mM Met, Met-RO, Met-SO, or Met-RSO at 0, 24, 48, 72, and 96 hours. FIG. 8D details a Western blot analysis of CBS in fRMsr-expressing SK-Hep1 cells grown in media containing 0.1 mM Met-RO or Met-SO at 0, 24, 48, 72, and 96 hours. FIG. 8E is a Western blot analysis of ATF3 in SK-Hep1 cells grown in media with 0.1 mM Met, Met-RO, or Met-SO at 72 hours. FIG. 8F details a Western blot analysis of ATF3 in fRMsr-expressing SK-Hep1 cells grown in media containing 0.1 mM Met, Met-RO or Met-SO at 72 hours.

Although levels of MsrA slightly decreased after 96 hours growth of SK-Hep1 cells, this change was observed in all samples and a significant difference in MsrA expression among the samples examined was not found. Thus, MsrA expression was not influenced by Met sulfoxide levels in cell culture media. The effect of addition of Met sulfoxides on regulation of selenoproteins expression was also tested as many of these proteins are important antioxidant enzymes or redox regulators (FIG. 8B). Metabolically labeled SK-Hep1 cells were used with ⁷⁵Se to examine selenoprotein patterns. No difference was observed among cells grown on Met, Met-RO, Met-SO and Met-RSO media.

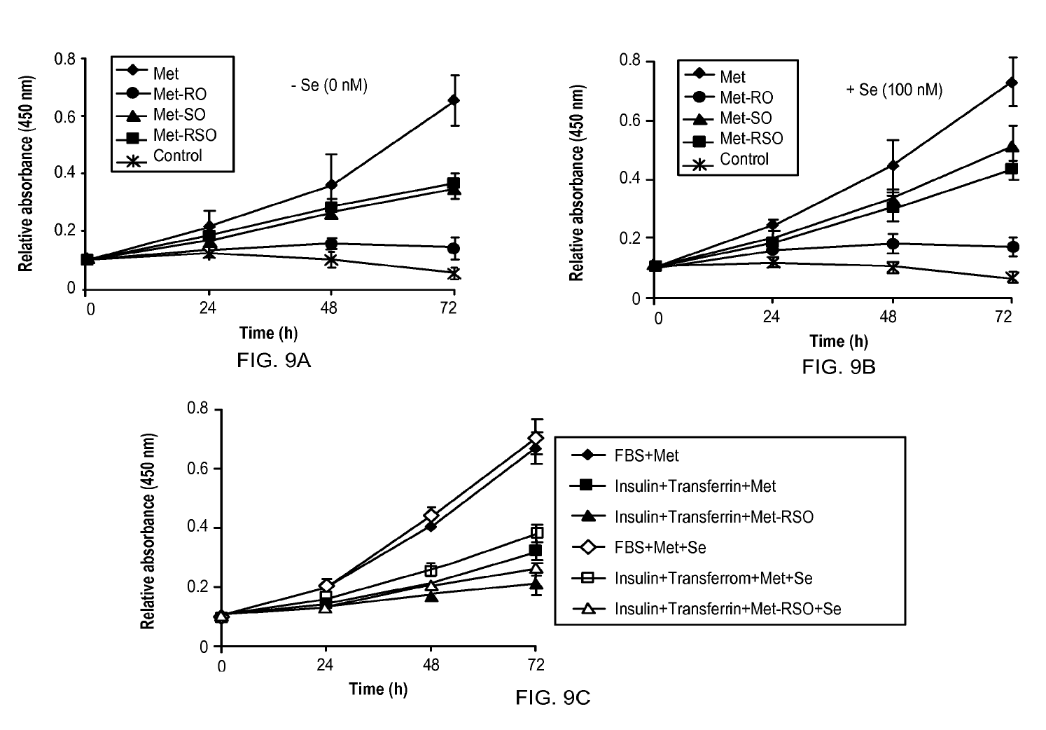

Addition of selenium (100 nM sodium selenite) also did not influence cell growth on Met and Met sulfoxide in FBS-supplemented and insulin/transferrin (FIG. 9) media. These data further argue against the role of selenoprotein MsrB1, which is a major peptide Met-RO reductase in mammals, in the reduction of free Met sulfoxides.

The expression of CBS in cells grown on Met and Met sulfoxide media was examined (FIGS. 9C and D). In the presence of Met-RO, SK-Hep1 cells reduced expression of this protein, whereas the cells grown on Met maintained stable CBS levels. In cells grown on the Met-SO medium, CBS levels were decreased, suggesting that although MsrA supports proliferation of SK-Hep1 cells by providing them with Met, these cells still suffer from Met deficiency (FIG. 9C). Interestingly, fRMsr-expressing cells grown on Met-RO had normal CBS levels (FIG. 9D). Thus, fRMsr can fully compensate for the lack of Met-RO reductase activity in SK-Hep1 hepatocytes and provide these cells with full amount of Met needed for cellular metabolism.

SK-Hep1 cells grown on Met-RO for 72 hours showed low expression levels of ATF3 as compared with cells on Met or Met-SO (FIG. 9E), suggesting that the cells grown on Met-RO had metabolic and proliferative defects due to Met limitation. Importantly, SK-Hep1 cells being quiescent on Met-RO recovered normal metabolic function and the ability to proliferate when transfected with a construct coding for yeast fRMsr (FIGS. 6B and 9F).

### Example 9

### Met-SO and Met-RO Levels in Mouse Plasma

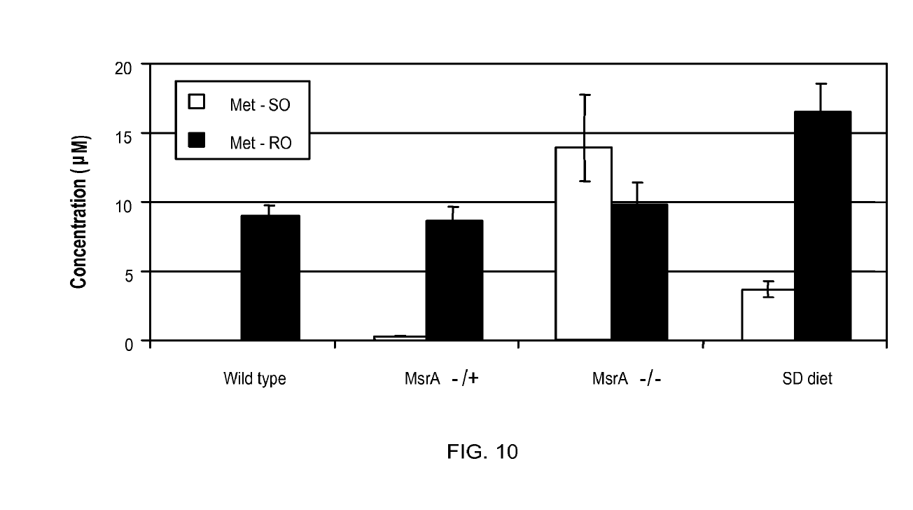

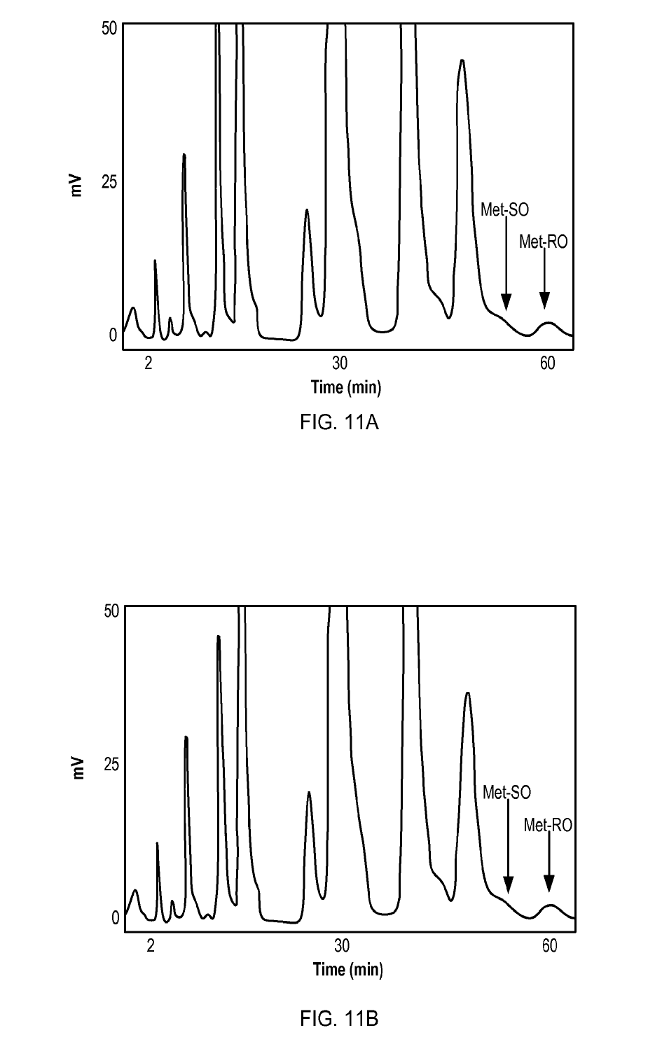

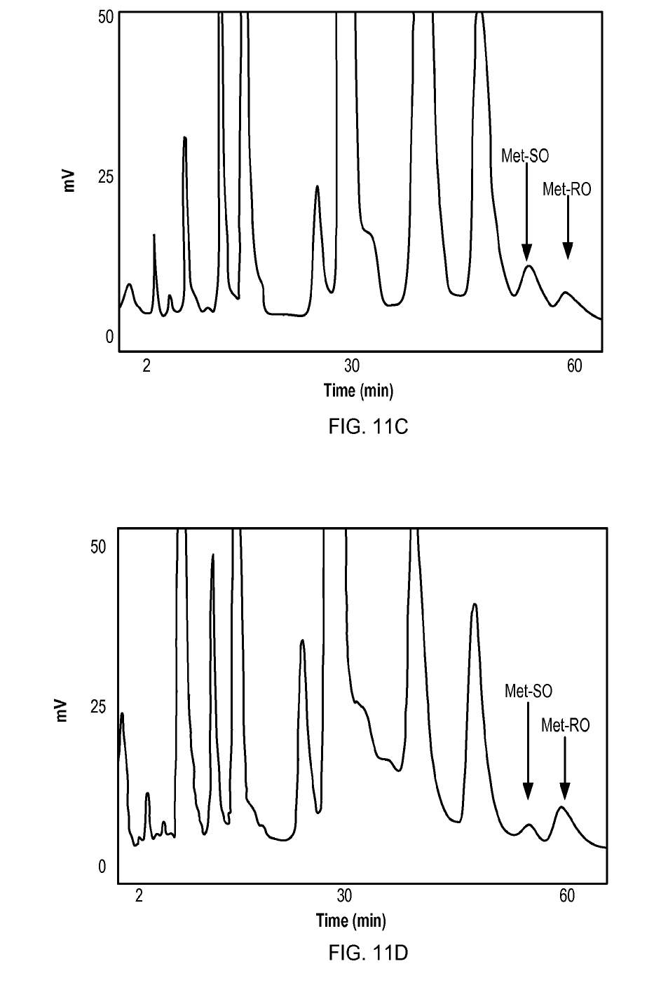

To test if differences in Met-SO and Met-RO consumption and reduction that were evident in cell culture experiments are also observed in an animal system, Met-SO and Met-RO levels in mouse plasma were examined. Met-SO was not detected in plasma of wild type mice, whereas Met-RO concentration was ≈9.1±0.7 μM (quantified by providing known amounts of Met-RO and Met-SO standards to plasma samples) (FIGS. 10 and 11A). To test if MsrA is responsible for low systemic Met-SO in mouse blood, samples from MsrA knockout mice were examined. These mice had similar levels of Met-SO and Met-RO in plasma (FIGS. 10 and 11B). Met-SO concentration was determined to be ≈14.6±3.1 μM and Met-RO was ≈9.8±1.6 μM (FIGS. 10 and 11C). These data show that deletion of MsrA caused a remarkable increase specifically in Met-SO levels, and indicated that MsrA is responsible for low levels of Met-SO in plasma of wild type mice. Wild type mice were also subjected to a selenium deficiency, which reduced MsrB1 to almost undetectable levels in the liver. Under these conditions, plasma Met-SO was ≈3.7±0.6 μM and Met-RO was ≈16.2±2.4 μM (FIGS. 10 and 11D), indicating that both sulfoxides showed slightly increased levels. Besides MsrB1, several selenoproteins serve as antioxidant proteins and show reduced expression in selenium deficiency, resulting in increased oxidative stress. Thus, slight elevation in both Met-SO and Met-RO under conditions of low dietary selenium could be due to an overall increased oxidative stress caused by systemic selenoprotein deficiency.

### Example 10

### R,S-sulforaphane

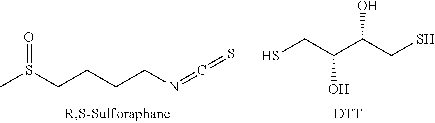

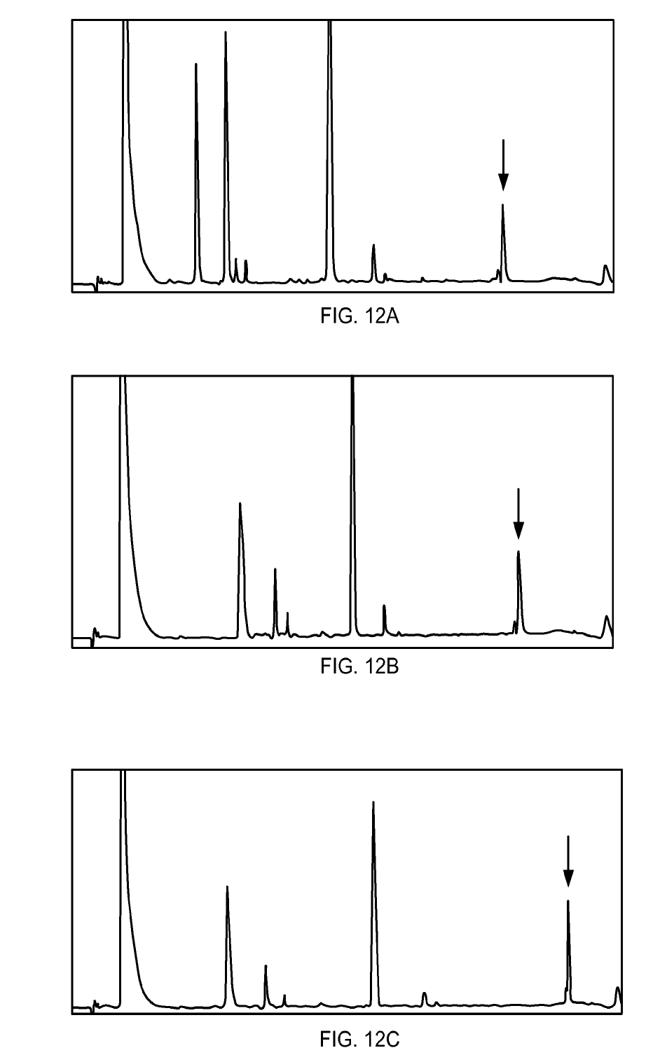

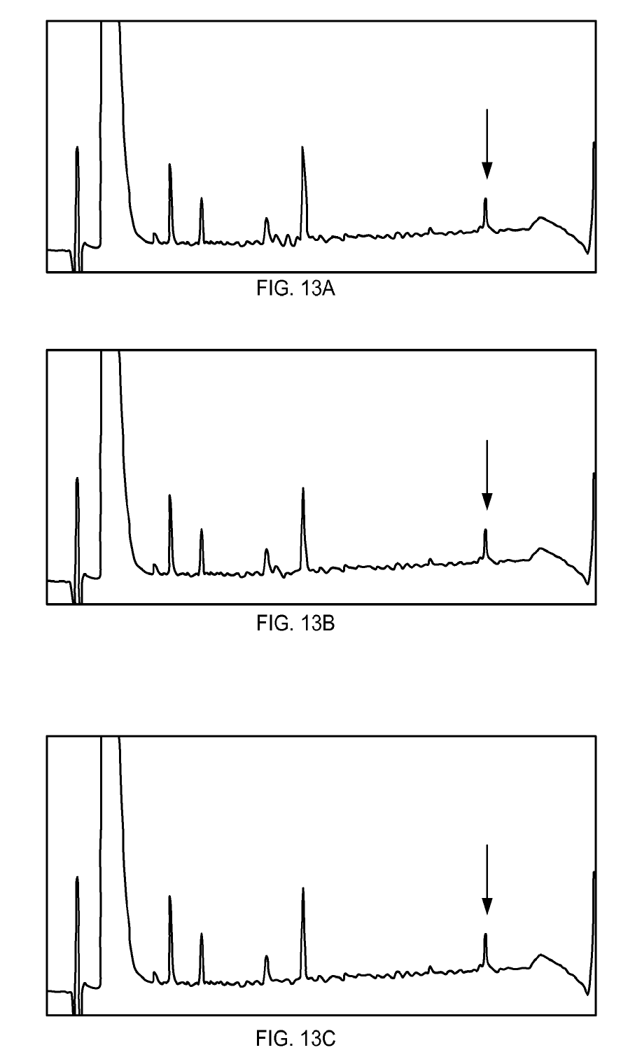

The isothiocyanate group in R,S-sulforaphane reacts with sulfhydryls to form conjugated compounds. First, the compound was treated with excess DTT (which has two free sulfhydryl groups) to conjugate the isothiocyanate group of R,S-sulforaphane or S-sulforaphane with the sulfhydryl group of DTT and the resulting adducts were then used as substrates for mouse MsrA and mouse MsrB2. The reduction of the DTT-sulforaphane adduct was analyzed using HPLC analysis of the adduct with buffer (as control; FIG. 12A), mouse MsrB2 (FIG. 12B), and mouse MsrA (FIG. 12C). The arrows indicate the migration of the DTT-erucin adduct. The data show that the DTT-S-sulforaphane adduct can be reduced by mouse MsrA, but not by mouse MsrB2 (although in each case partial reduction is observed with DTT alone). FIG. 13 illustrates the HPLC analysis of the DTT-R-sulforaphane adduct incubated with buffer (as control) (FIG. 13A), mouse MsrA (FIG. 13B), and mouse MsrB2 (FIG. 13C). Again, the arrows show the migration of the DTT-erucin adduct. The data show that the DTT-R-sulforaphane adduct cannot be reduced by both of mouse MsrA and mouse MsrB2 (although in each case partial reduction is observed with DTT alone).

### Example 11

### Mesoridazine (Serentil)

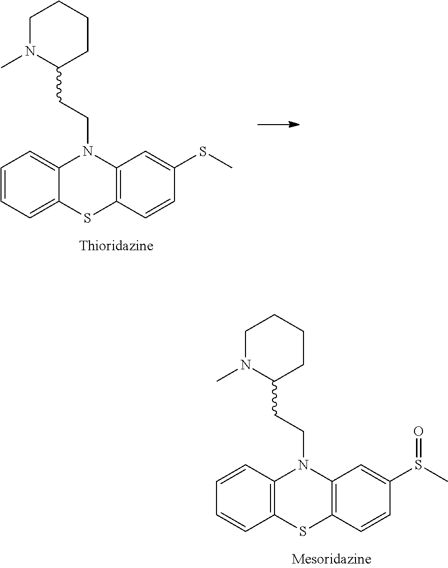

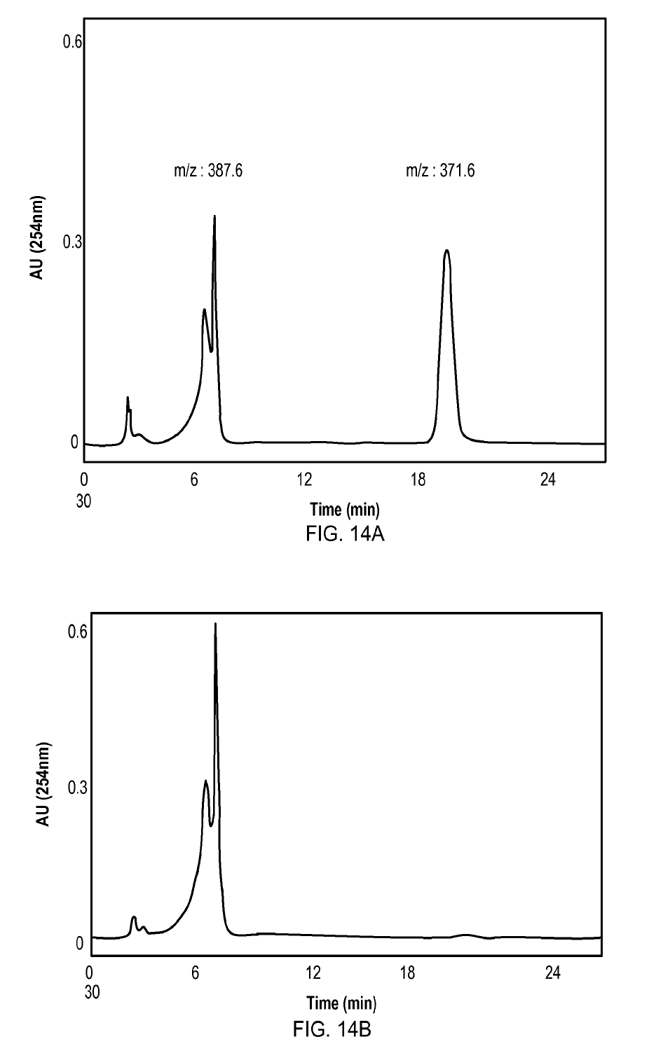

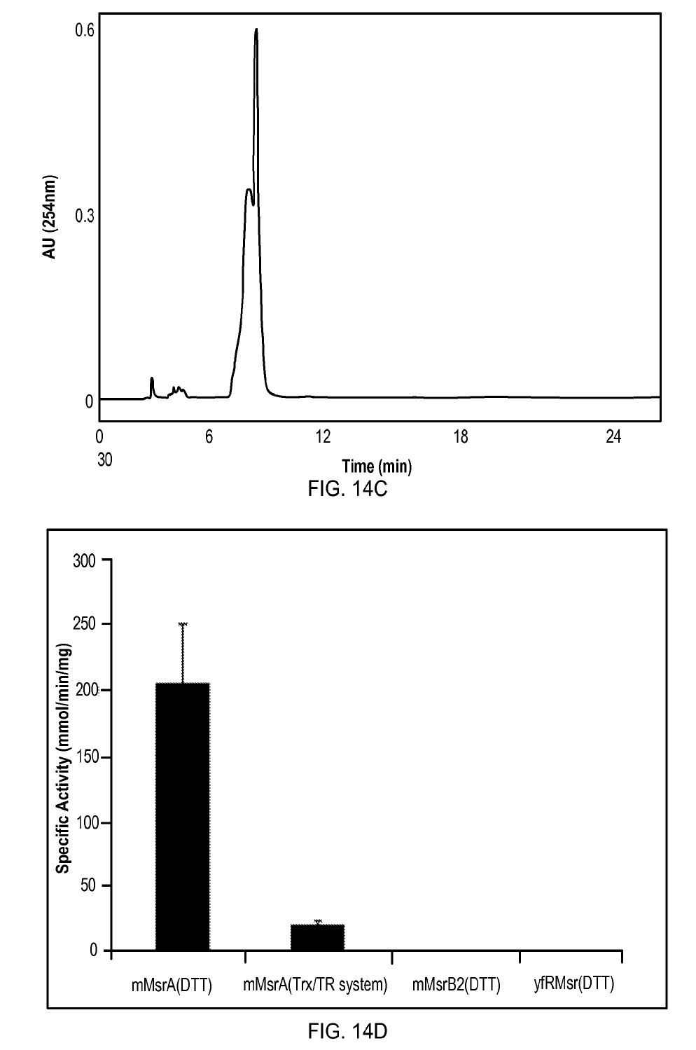

Mesoridazine, which has a mild effect on treating schizophrenia, was examined to determine its reduction by yeast fRMsr, mouse MsrB2, and mouse MsrA (FIG. 14). The HPLC analysis of mesoridazine incubated with mouse MsrA and DTT is shown in FIG. 14A. The peak of m/z 387.6 corresponds to the molecular weight of mesoridazine and the peak of m/z 371.6 corresponds to the molecular weight of thioridazine. FIG. 14B details the HPLC analysis of mesoridazine incubated with mouse MsrB2 and DTT. FIG. 14C provides the HPLC analysis of mesoridazine incubated with yeast fRMsr and DTT. FIG. 14D illustrates the specific activities of various Msrs for the reduction of mesoridazine (left to right): mouse MsrA in the presence of DTT as a reductant, mouse MsrA in the presence of the thioredoxin reducing system, mouse MsrB2 in the presence of DTT, and yeast fRMsr in the presence of DTT. Only MsrA showed the ability to reduce mesoridazine. Mesoridazine was reduced to thioridazine by mouse MsrA with either DTT or thioredoxin system as reductants, but no activity was observed with mouse MsrB2 or yeast fRMsr.

### Example 12

### Triclabendazole (Fasinex)

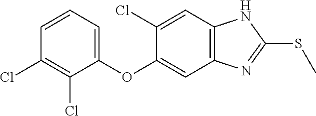

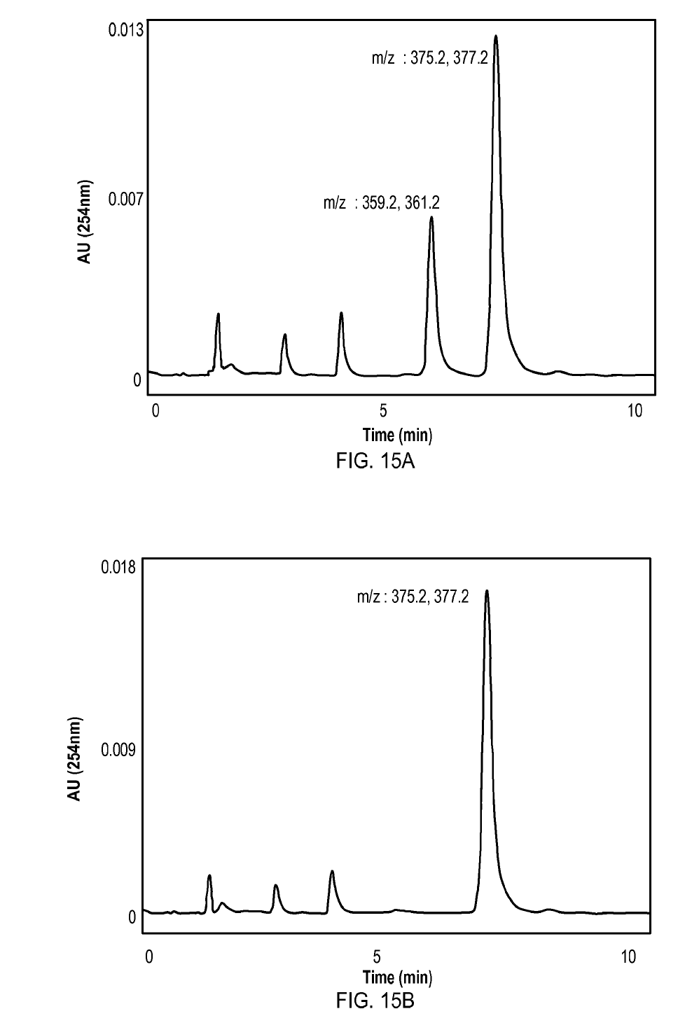

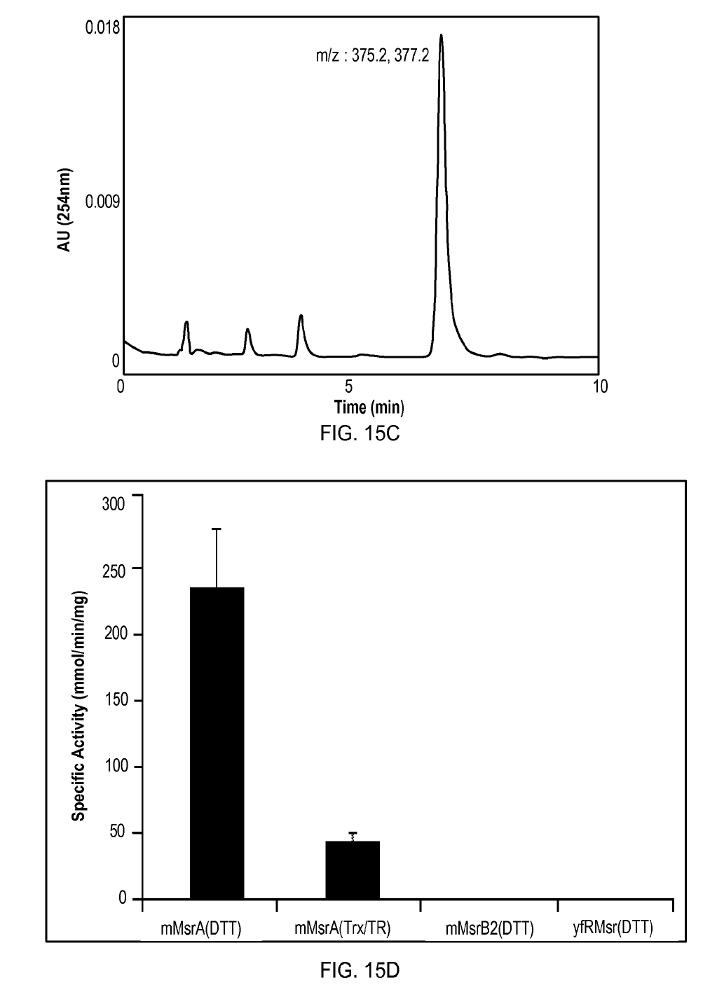

The reduction of triclabendazole sulfoxide was examined. This compound has been used to treat liver fluke, Fasciola hepatica and Fasciola gigantica, in sheep, goat, and cattle as a form of triclabendazole. Triclabendazole can be metabolized to several different forms including triclabendazole sulfoxide, which is an oxidized form of methylsulfide group, in biological systems. Reduction of triclabendazole sulfoxide by mouse MsrA, mouse MsrB2, and yeast fRMsr is shown in FIG. 15. FIG. 15A provides an HPLC analysis of triclabendazole sulfoxide incubated with mouse MsrA and DTT. The peak of m/z 375.2 and 377.2 correspond to the molecular weight of triclabendazole sulfoxide and the peak of m/z 359.2 and 361.2 correspond to the molecular weight of triclabendazole. FIG. 15B illustrates an HPLC analysis of triclabendazole sulfoxide incubated with mouse MsrB2 and DTT. FIG. 15C details an HPLC analysis of triclabendazole sulfoxide incubated with yeast fRMsr and DTT. FIG. 15D illustrates the specific activities of various Msrs for the reduction of triclabendazole sulfoxide (left to right): mouse MsrA in the presence of DTT as a reductant, mouse MsrA in the presence of the thioredoxin reducing system, mouse MsrB2 in the presence of DTT, and yeast fRMsr in the presence of DTT. Only MsrA showed the ability to reduce triclabendazole sulfoxide. It was determined that triclabendazole sulfoxide is reduced back to triclabendazole by mouse MsrA with either DTT or thioredoxin system as reductants, but it was not reduced by mouse MsrB2 or yeast fRMsr.

### Example 13

### Sulmazole

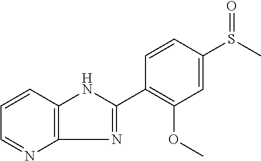

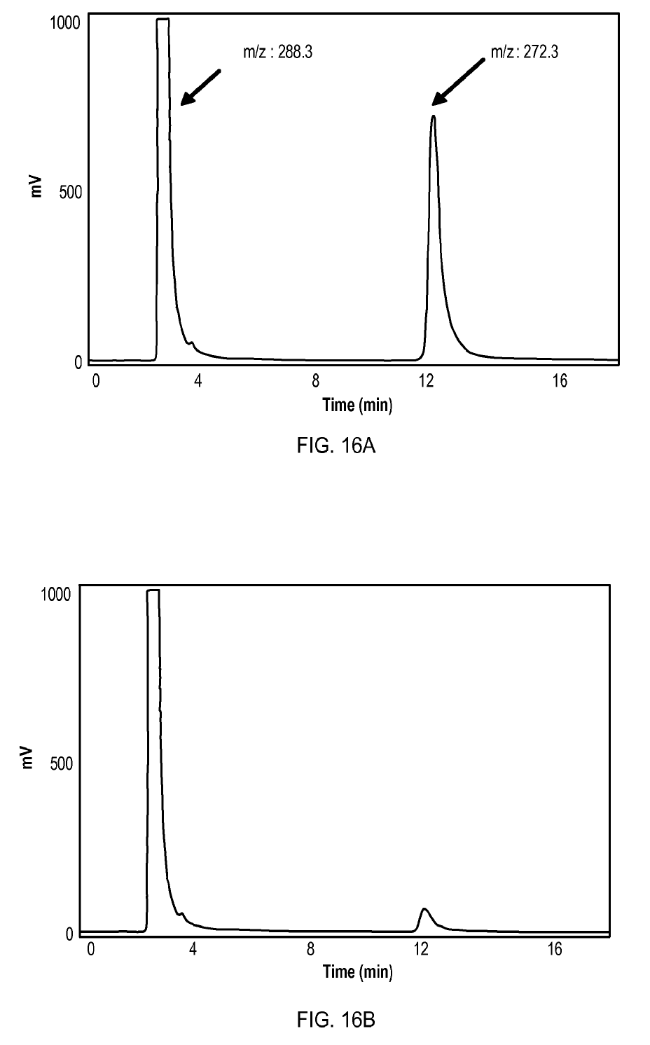

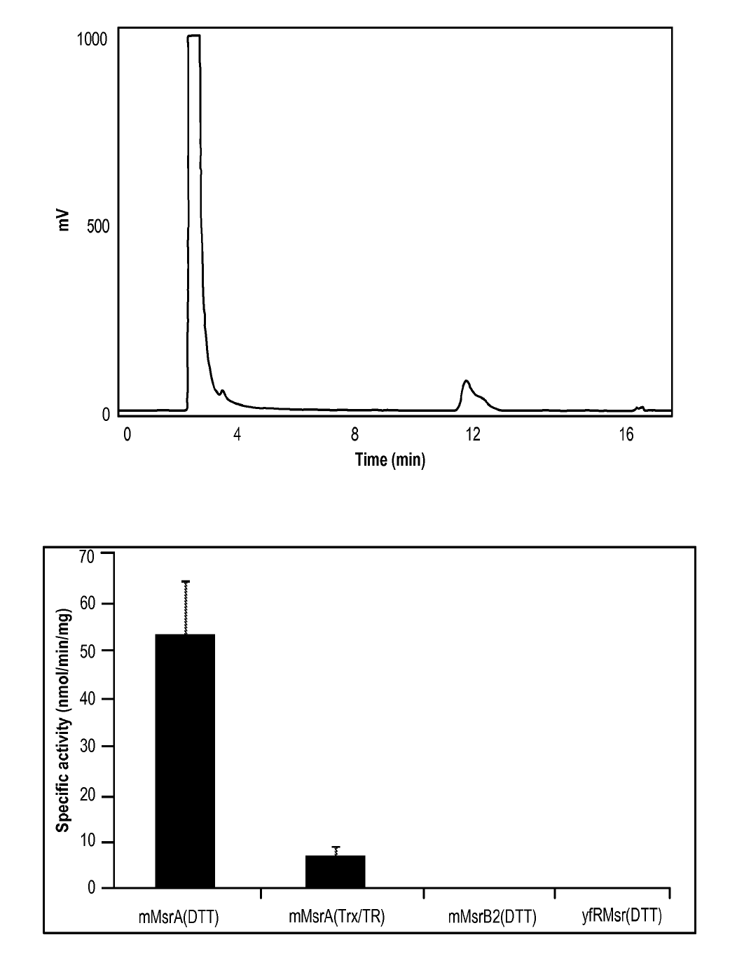

Another methylsulfinyl-containing drug, sulmazole, was tested as a substrate for Msrs. This drug is pharmacologically active as a cardiotonic agent and is a phosphodiesterase inhibitor. Reduction of sulmazole by mouse MsrA, mouse MsrB2, and yeast fRMsr is shown in FIG. 16. FIG. 16A details an HPLC analysis of sulmazole incubated with mouse MsrA and DTT. The peak of m/z 288.3 corresponds to the molecular weight of sulmazole and the peak of m/z 272.3 corresponds to the molecular weight of reduced form of sulmazole. FIG. 16B illustrates an HPLC analysis of sulmazole incubated with mouse MsrB2 and DTT. FIG. 16C provides an HPLC analysis of sulmazole incubated with yeast fRMsr and DTT. FIG. 16D details the specific activities of various Msrs for the reduction of sulmazole (left to right): mouse MsrA in the presence of DTT as a reductant, mouse MsrA in the presence of the thioredoxin reducing system, mouse MsrB2 in the presence of DTT, and yeast fRMsr in the presence of DTT. Only MsrA showed the ability to reduce sulmazole. Based on the HPLC analysis, the methylsulfinyl moiety of this drug is specifically reduced by mouse MsrA with either DTT or thioredoxin system, but it was not reduced by mouse MsrB2 or yeast fRMsr.

### Example 14

### DMSO (Dimethylsulfoxide)

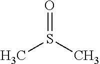

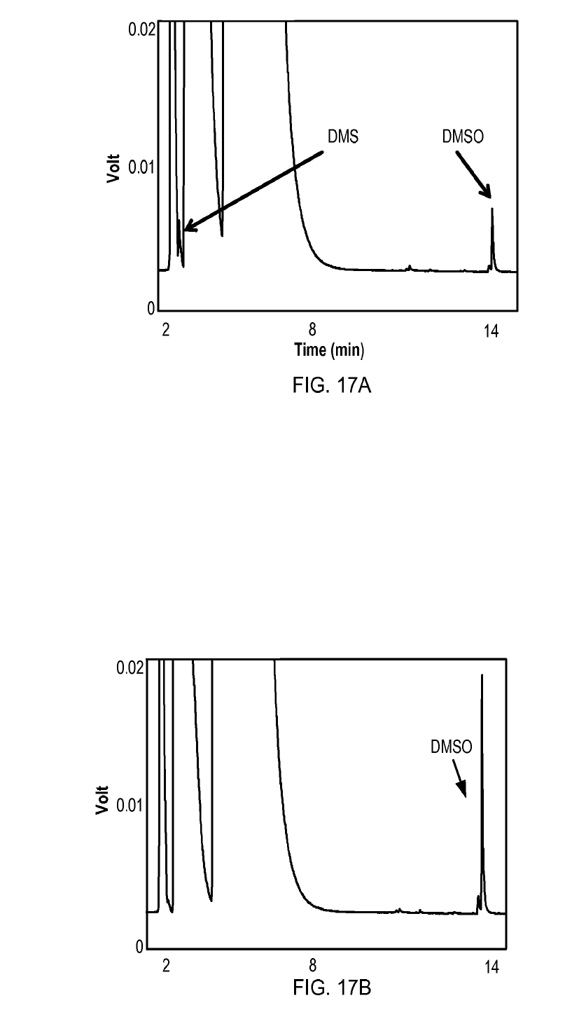

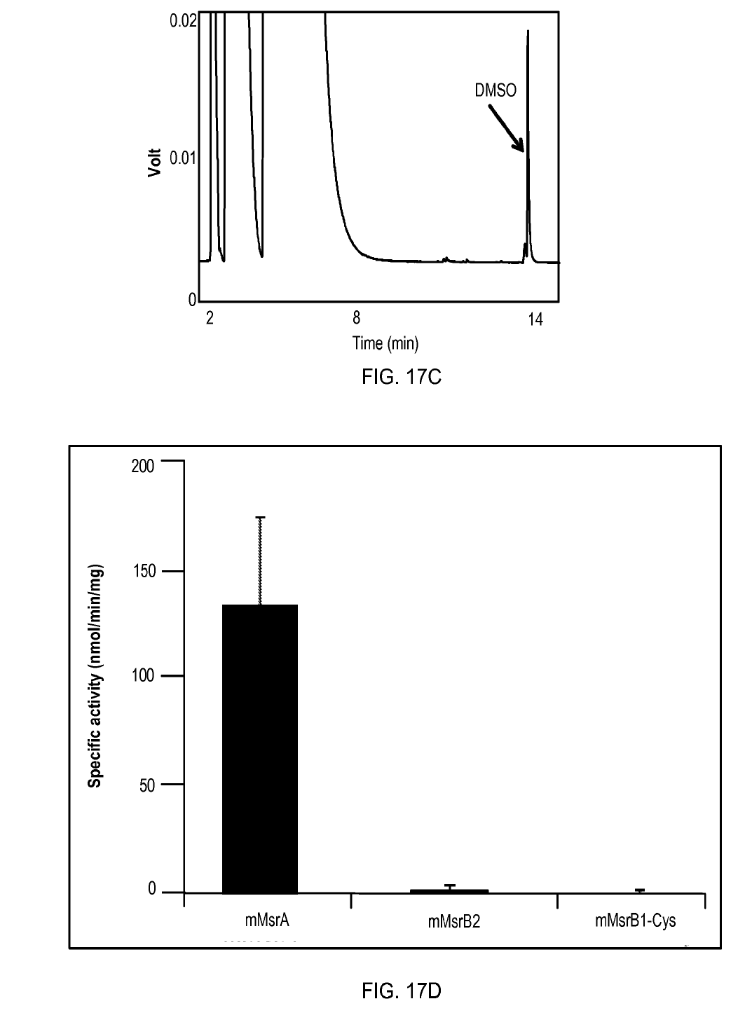

DMSO, which is used as a preservative for stem cells, solvent for drugs, and a drug itself with anti-inflammatory and free radical scavenging properties, was studied for its reduction by Msrs. Reduction of DMSO to DMS by mouse MsrA, mouse MsrB2, and mouse MsrB1-Cys is shown in FIG. 17. FIG. 17A details a GC analysis of DMSO incubated with mouse MsrA and DTT. FIG. 17B provides a GC analysis of DMSO incubated with mouse MsrB2 and DTT. FIG. 17C illustrates a GC analysis of DMSO incubated with mouse MsrB1-Cys and DTT. FIG. 17D provides specific activities of various Msrs for the reduction of DMSO (left to right): mouse MsrA in the presence of DTT as a reductant, mouse MsrB2 in the presence of DTT, and mouse MsrB1-Cys in the presence of DTT. Only MsrA showed the ability to reduce DMSO. Interestingly, this compound can be reduced by mouse MsrA with DTT, but is a very inefficient substrate for mouse MsrB2 and mouse mutant MsrB1, in which selenocysteine was replaced with cysteine.

### Example 15

### Survey of Genomic Databases for fRMsr

S. cereivisiae and E. coli fRMsr protein sequences were used as query sequences to search completely sequenced genomes for fRMsr genes using the TBLASTN program with default parameters (see ncbi.nlm.nih.gov on the World Wide Web). fRMsrs were detected in a variety of prokaryotes and unicellular eukaryotes, but they were absent in the genome of all animals examined including, for example, in Caenorhabditis elegans, Drosophila melanogaster, Danio rerio, Xenopus, Gallus gallus and 20 examined mammalian genomes. Thus, fRMsr is apparently absent in all animals, indicating a deficiency in the ability to reduce the R epimer of methylsulfinyl-containing compounds in these organisms.

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating a subject with a drug comprising a methylsulfinyl moiety comprising:
a) determining whether the drug comprising the methylsulfinyl moiety exhibits higher biological activity when the methylsulfinyl moiety is present in the methylsulfinyl-oxidized form or the methylsulfide-reduced form; and
b) administering, to the subject, a composition comprising the drug in an excess amount of the R-epimer relative to the S-epimer if the methylsulfinyl-oxidized form exhibits higher biological activity, or a composition comprising the drug in an excess amount of the S-epimer relative to the R-epimer if the methylsulfide-reduced form exhibits higher biological activity.

2. The method of claim 1, wherein the drug is chosen from: enoximone; pergolide; lincomycin; thiethylperazine; fensulfothion; nifuratel; albendazole; modafinil; captodiame; sulfinpyrazone; clindamycin; thiocolchicoside; omeprazole; flosequinan; dimethylsulfoxide; sulmazole; triclabendazole; mesoridazine; oxisuran; and sulindac.

3. The method of claim 1, wherein the amount of the drug in the R-epimer form is at least 75% by weight compared to the S-epimer.

4. The method of claim 1, wherein the amount of the drug in the R-epimer form is at least 90% by weight compared to the S-epimer.

5. The method of claim 1, wherein the amount of the drug in the S-epimer form is at least 75% by weight compared to the R-epimer.

6. The method of claim 1, wherein the amount of the drug in the S-epimer form is at least 90% by weight compared to the R-epimer.

7. The method of claim 1, wherein the drug is administered with a pharmaceutically acceptable carrier or diluent.

8. The method of claim 7, wherein the pharmaceutically acceptable carrier or diluent has a methylsulfinyl moiety if the methylsulfinyl-oxidized form exhibits higher biological activity.

9. The method of claim 7, wherein the pharmaceutically acceptable carrier or diluent does not have a methylsulfinyl moiety if the methylsulfide-reduced form exhibits higher biological activity.

10. A method for increasing the shelf-stability of a drug comprising a methylsulfinyl moiety comprising:
a) determining whether the drug comprising the methylsulfinyl moiety exhibits higher biological activity when the methylsulfinyl moiety is present in the methylsulfinyl-oxidized form or the methylsulfide-reduced form; and
b) formulating
(i) a composition comprising the drug in an excess amount of the R-epimer relative to the S-epimer if the methylsulfinyl-oxidized form exhibits higher biological activity and an oxidant, or
(ii) a composition comprising the drug in an excess amount of the S-epimer relative to the R-epimer if the methylsulfide-reduced form exhibits higher biological activity and a reductant.

11. The method of claim 10, wherein the oxidant is chosen from hydrogen peroxide, hypochlorous acid, urea peroxide, sodium perborate tetrahydrate, sodium percarbonate, sodium perborate, sodium peroxide, sodium periodate, calcium peroxide, and mixtures thereof.

12. The method of claim 10, wherein the reductant is chosen from dithiothreitol (DTT), a thioredoxin, sodium dithionite, sodium bisulphite, ascorbic acid, sodium ascorbate, calcium ascorbate, palmityl-DL-ascorbic acid, propyl gallate, octyl gallate, dodecyl gallate, butylhydroxyanisole gallate and butylhydroxytoluene gallate, formamidine sulphinic acid, stannous ion, Fe(II), Cu(I), erythrobate, α-tocopherol, γ-tocopherol, δ-tocopherol, oxalic acid, formic acid, and mixtures thereof.

13. A method for increasing the in vivo activity of a drug comprising a methylsulfinyl moiety in a subject comprising:
a) determining whether the drug comprising the methylsulfinyl moiety exhibits higher biological activity when the methylsulfinyl moiety is present in the methylsulfinyl-oxidized form or the methylsulfide-reduced form; and
b) administering, to the subject,
(i) an oxidant and a composition comprising the drug in an excess amount of the R-epimer relative to the S-epimer if the oxidized form exhibits higher biological activity; or
(ii) a reductant and a composition comprising an excess amount of the S-epimer relative to the R-epimer if the reduced form exhibits higher biological activity.

14. The method of claim 13 wherein the oxidant or the reductant is administered before or after the drug.

15. The method of claim 13, wherein the oxidant or the reductant and the drug are administered together.

16. The method of claim 13, wherein the oxidant or reductant is formulated into the composition comprising the drug.

17. The method of claim 13, wherein the oxidant is chosen from hydrogen peroxide, hypochlorous acid, urea peroxide, sodium perborate tetrahydrate, sodium percarbonate, sodium perborate, sodium peroxide, sodium periodate, calcium peroxide, and mixtures thereof.

18. The method of claim 13, wherein the reductant is chosen from dithiothreitol (DTT), a thioredoxin, sodium dithionite, sodium bisulphite, ascorbic acid, sodium ascorbate, calcium ascorbate, palmityl-DL-ascorbic acid, propyl gallate, octyl gallate, dodecyl gallate, butylhydroxyanisole gallate and butylhydroxytoluene gallate, formamidine sulphinic acid, stannous ion, Fe(II), Cu(I), erythrobate, α-tocopherol, γ-tocopherol, δ-tocopherol, oxalic acid, formic acid, and mixtures thereof.

19. A composition wherein a drug comprising a methylsulfinyl moiety exhibits higher biological activity when the methylsulfinyl moiety is present in the methylsulfinyl-oxidized form comprising:
a) the drug in an excess amount of the R-epimer relative to the S-epimer; and
b) an oxidant.

20. The method of claim 19, wherein the oxidant is chosen from hydrogen peroxide, hypochlorous acid, urea peroxide, sodium perborate tetrahydrate, sodium percarbonate, sodium perborate, sodium peroxide, sodium periodate, calcium peroxide, and mixtures thereof.

21. A composition wherein a drug comprising a methylsulfinyl moiety exhibits higher biological activity when the methylsulfinyl moiety is present in the methylsulfide-reduced form comprising:
c) the drug in an excess amount of the S-epimer relative to the R-epimer; and
d) a reductant.

22. The method of claim 21, wherein the reductant is chosen from dithiothreitol (DTT), a thioredoxin, sodium dithionite, sodium bisulphite, ascorbic acid, sodium ascorbate, calcium ascorbate, palmityl-DL-ascorbic acid, propyl gallate, octyl gallate, dodecyl gallate, butylhydroxyanisole gallate and butylhydroxytoluene gallate, formamidine sulphinic acid, stannous ion, Fe(II), Cu(I), erythrobate, α-tocopherol, γ-tocopherol, δ-tocopherol, oxalic acid, formic acid, and mixtures thereof.

23. A method of treating a subject with a drug comprising a methylsulfinyl moiety comprising:
e) determining whether the drug comprising the methylsulfinyl moiety exhibits higher biological activity when the methylsulfinyl moiety is present in the methylsulfinyl-oxidized form or the methylsulfide-reduced form; and
f) administering, to the subject, a composition comprising the drug and a pharmaceutically acceptable carrier or diluent having a methylsulfinyl moiety, if the methylsulfinyl-oxidized form exhibits higher biological activity, or a composition comprising the drug and a pharmaceutically acceptable carrier or diluent lacking a methylsulfinyl moiety, if the methylsulfide-reduced form exhibits higher biological activity.

24. A method of treating a subject with a compound comprising a methylsulfinyl moiety comprising:
g) determining whether the compound comprising the methylsulfinyl moiety exhibits higher biological activity when the methylsulfinyl moiety is present in the methylsulfinyl-oxidized form or the methylsulfide-reduced form; and
h) contacting the subject with a composition comprising the compound in an excess amount of the R-epimer relative to the S-epimer if the methylsulfinyl-oxidized form exhibits higher biological activity, or a composition comprising the compound in an excess amount of the S-epimer relative to the R-epimer if the methylsulfide-reduced form exhibits higher biological activity.
